(19) United States
(12) Patent Application Publication
(10) Pub. No.: US 2026/0232429 A1
(43) Pub. Date: Aug. 13, 2026

(54) DEVICES AND METHODS OF MAKING AND USE THEREOF

(71) Applicant: THE METHODIST HOSPITAL, Houston, TX (US)

(72) Inventor: Warren Aldrich ELLSWORTH IV, Houston, TX (US)

(21) Appl. No.: 19/471,443

(22) PCT Filed: Apr. 3, 2024
(86) PCT No.: PCT/US2024/022710

§ 371 Date: Oct. 1, 2025

Related U.S. Application Data
(60) Provisional application No. 63/456,629, filed on Apr. 3, 2023.

(51) Int. Cl. A61F 2/12

(52) U.S. Cl. CPC A61F 2/12; A61F 2210/0004; A61F 2220/0075

(57) ABSTRACT

Disclosed herein are devices for facilitating reconstruction, augmentation, and/or cosmetic improvement of a breast.

### CROSS-REFERENCE TO RELATED APPLICATIONS

This application claims the benefit of priority to U.S. Provisional Application No. 63/456,629, filed Apr. 3, 2023, which is hereby incorporated herein by reference in its entirety.

### BACKGROUND

There is currently limited technology to treat breast reconstruction, in particular implant-based breast reconstruction, appropriately. In particular, there is a lack of technology that can both support breast implants in breast reconstruction and offer soft tissue coverage of the most projecting portion and the upper pole of the implant. The devices and methods discussed herein address these and other needs.

### SUMMARY

In accordance with the purposes of the disclosed devices and methods as embodied and broadly described herein, the disclosed subject matter relates to devices and methods of making and use thereof. For example, described herein are devices for facilitating reconstruction, augmentation, and/or cosmetic improvement of a breast. The devices can, for example, comprise a dual synthetic and biologic design for breast reconstruction, augmentation, and/or cosmetic improvement.

For example, disclosed herein are devices for facilitating reconstruction, augmentation, and/or cosmetic improvement of a breast, the breast having an upper pole, a lower pole, a most projecting portion, and an inframammary fold. In some examples, the devices comprise a superior portion comprising a biological matrix sheet, the superior portion having a lower end positioned towards the most projecting portion, the superior portion being configured to extend superiorly from the most projecting portion of the breast and form a slope extending along the upper pole of the breast. In some examples, the devices further comprise an inferior portion comprising a synthetic mesh sheet, the inferior portion having an upper end positioned towards the most projecting portion, the inferior portion being configured to extend inferiorly from the upper end and form an inward curvature extending along the lower pole of the breast towards the inframammary fold. In some examples, the devices further comprise a connection portion configured to connect the lower end of the superior portion to the upper end of the inferior portion.

In some examples, at least a portion of the connection portion is positioned at or below the most projecting portion of the breast. In some examples, the connection portion is a seam extending between the lower end and the upper end. In some examples, the superior portion is contiguous with the inferior portion along the connection portion.

In some examples, the lower end includes a lower edge, the upper end includes an upper edge, and the lower edge is connected to the upper edge to form at least a portion of the connection portion.

In some examples, the superior portion and the inferior portion overlap along the connection portion. In some examples, the superior portion and the inferior portion overlap by a distance of from 0.1 centimeter (cm) to 10 cm along the connection portion.

In some examples, the connection portion is straight, curved, or a combination thereof. In some examples, the connection portion comprises a first section and a second section, wherein the first section and the second section each independently is straight or curved.

In some examples, the inferior portion further includes a lateral portion extending at least partially into the upper pole.

In some examples, the superior portion is connected to the inferior portion by sutures, staples, glue, weaving, or a combination thereof. In some examples, the superior portion is sewn to the inferior portion with a permanent suture and/or a biodegradable suture. In some examples, the superior portion is sewn to the inferior portion with a single suture line.

In some examples, the biological matrix sheet comprises an acellular tissue matrix (ATM), such as an acellular dermal matrix (ADM). In some examples, the biological matrix sheet is derived from human dermis, animal dermis, or animal material. In some examples, the biological matrix sheet comprises FlexHD pliable ADM, FlexHD pliable MAX ADM, AlloDerm®, DermACELL (e.g., DermACELL AWM®), etc.), porcine ADM, STRATTICE®, CORTIVA™ (e.g., CORTIVA™ allograft dermis), ALLOMAX™, SurgiMend®, ARTIA™, or a combination thereof.

In some examples, the superior portion is perforated (e.g., by a plurality of perforations). In some examples, the synthetic mesh sheet is biodegradable. In some examples, the synthetic mesh sheet comprises vicryl mesh, gortex, poly(4-Hydroxy buterate) (e.g., Galaflex), Polydioxanone (PDO) (e.g., Durasorb), polyglycolic acid trimethylene carbonate (PGA-PMC) (e.g., TIGR matrix), silk (e.g., SERI scaffold), polypropylene and/or polyglycolic acid (e.g., OviTex®), or a combination thereof. In some examples, the synthetic mesh sheet has a strength profile of from 15 Newtons per centimeter (N/cm) to 150 N/cm. In some examples, the synthetic mesh sheet has an initial burst strength of from 50 Newtons (N) to 350 N.

In some examples, the device has a surface area, from 40 to 60% of the surface area of the device is the superior portion, from 40 to 60% of the surface area of the device is the inferior portion, the superior portion and the inferior portion together comprising 90% or more of the surface area of the device.

In some examples, the device is symmetric in shape. In some examples, the device is asymmetric in shape. In some examples, the device has a shape that is substantially circular, ovate, ovoid, elliptic, or teardrop.

In some examples, the superior portion has a first edge, the inferior portion has a second edge, and the device has a periphery comprising at least a portion of the first edge and the second edge. In some examples, at least a portion of the first edge, the second edge, the periphery, or a combination thereof is curved.

In some examples, the device is reversible (e.g., posterior and anterior surfaces are substantially the same).

In some examples, the device has an average characteristic dimension (e.g., diameter) of from 7 centimeters to 25 centimeters.

In some examples, the superior portion, the inferior portion, the device, or a combination thereof is substantially flat. In some examples, the superior portion, the inferior portion, the device, or a combination thereof is shaped, such as curved or rounded (e.g., cup shaped).

In some examples, the device is configured to be utilized with a breast implant or tissue expander. In some examples, the device is configured to substantially cover the breast implant or tissue expander. In some examples, in use, the device is positioned prepectorally or postpectorally, anterior to the breast implant or tissue expander, such that the superior portion of the device is positioned superiorly (towards the upper pole) and the inferior portion of the device is positioned inferiorly (towards the lower pole), and wherein the superior portion, the inferior portion, or the connection portion of the device is positioned anterior to the most projecting portion of the breast implant or tissue expander. In some examples, the size and/or shape of the superior portion, the inferior portion, the device, or a combination thereof is selected based on the volume and/or shape of the breast implant or tissue expander. In some examples, the breast implant or tissue expander has an intra-operative fill volume of from 0 cubic centimeters (cc) to 1500 cc.

In some examples, the device further comprises one or more (e.g., 2 or more, 3 or more, 4 or more, or 5 or more) steps of synthetic material, each of the one or more strips extending from a portion of the second edge to a distal end. In some examples, the distal end of each of the one or more strips is configured to be attached to a portion of the first edge, for example such that each of the one or more strips can at least partially encircle the breast, breast implant, and/or tissue expander. In some examples, the distal end of each of the one or more strips is attached to a portion of the first edge by sutures, staples, glue, weaving, or a combination thereof.

In some examples, the device further comprises a therapeutic agent dispersed within the inferior portion. In some examples, the therapeutic agent comprises an anticancer agent, anti-inflammatory agent, analgesic agent, antimicrobial agent, or a combination thereof. In some examples, the therapeutic agent comprises a chemotherapeutic agent, an immunotherapeutic agent, or a combination thereof.

In some examples, the device is implantable in a subject. In some examples, the device is anatomically designed for the subject.

In some examples, the inferior portion is configured to be stable for an amount of time of from 1 month to 5 years after the device is implanted in the subject, such as from 9 months to 5 years, or from 9 months to 2.5 years.

In some examples, the device is biocompatible.

Also disclosed herein are kits comprising any of the devices disclosed herein. For example, wherein the kit comprises the device disposed within a sterile package.

Also disclosed herein are methods of manufacturing any of the devices disclosed herein. For example, the methods comprise connecting the superior portion to the inferior portion. In some examples, the superior portion is connected to the inferior portion by sutures, staples, glue, weaving, or a combination thereof. In some examples, the superior portion is sewn to the inferior portion with a permanent suture and/or a biodegradable suture. In some examples, the superior portion is sewn to the inferior portion with a single suture line. In some examples, the method further comprises making the device based on a 3D model. In some examples, the 3D model is based on an anatomical image of a subject. In some examples, the method further comprises collecting the anatomical image of the subject.

Also disclosed herein are methods of treating a subject in need thereof, the methods comprising implanting any of the devices disclosed herein into the subject. In some examples, the device is implanted into a breast of the subject. In some examples, the method comprises breast reconstruction, augmentation, and/or cosmetic improvement. In some examples, the method comprises one stage or two stage implant based breast reconstruction. In some examples, the method further comprises anatomically designing the device for the subject.

Additional advantages of the disclosed devices and methods will be set forth in part in the description which follows, and in part will be obvious from the description. The advantages of the disclosed devices and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

The details of one or more embodiments of the invention are set forth in the accompanying drawings and the description below. Other features, objects, and advantages of the invention will be apparent from the description and drawings, and from the claims.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1. Schematic view of an example device as disclosed herein according to one implementation.

FIG. 2. Schematic view of an example device as disclosed herein according to one implementation.

FIG. 3. Schematic view of an example device as disclosed herein according to one implementation.

FIG. 4. Schematic view of an example device as disclosed herein according to one implementation.

FIG. 5. Schematic view of an example device as disclosed herein according to one implementation.

FIG. 6. Schematic view of an example device as disclosed herein according to one implementation.

FIG. 7. Schematic view of an example device as disclosed herein according to one implementation.

FIG. 8. Schematic view of an example device as disclosed herein according to one implementation.

FIG. 9. Schematic view of an example device as disclosed herein according to one implementation.

FIG. 10. Schematic view of an example device as disclosed herein according to one implementation.

FIG. 11. Schematic view of an example device as disclosed herein according to one implementation.

FIG. 12. Schematic view of an example device as disclosed herein according to one implementation.

FIG. 13. Schematic view of an example device as disclosed herein according to one implementation.

FIG. 14. Schematic view of an example device as disclosed herein according to one implementation.

FIG. 15. Schematic view of an example device as disclosed herein according to one implementation.

FIG. 16. Schematic cut away side view of an example device in use within a breast of a subject as disclosed herein according to one implementation.

FIG. 17. Schematic cut away side view of an example device in use within a breast of a subject as disclosed herein according to one implementation.

FIG. 18. Schematic cut away side view of an example device in use with a breast implant within a breast of a subject as disclosed herein according to one implementation.

FIG. 19. Schematic view of an example device as disclosed herein according to one implementation.

FIG. 20. Schematic view of an example device as disclosed herein according to one implementation.

FIG. 21. Schematic view of an example device as disclosed herein according to one implementation.

FIG. 22. Schematic view of an example device as disclosed herein according to one implementation.

FIG. 23. Schematic view of an example device as disclosed herein according to one implementation.

FIG. 24. Schematic view of an example device as disclosed herein according to one implementation.

FIG. 25. Schematic view of an example device as disclosed herein according to one implementation.

FIG. 26. Schematic view of an example device as disclosed herein according to one implementation.

FIG. 27. Schematic view of an example device as disclosed herein according to one implementation.

FIG. 28. Schematic view of an example device as disclosed herein according to one implementation.

FIG. 29. Schematic view of an example device as disclosed herein according to one implementation.

FIG. 30. Schematic view of an example device as disclosed herein according to one implementation.

FIG. 31. Schematic view of an example device as disclosed herein according to one implementation.

FIG. 32. Schematic view of an example device as disclosed herein according to one implementation.

FIG. 33. Schematic view of an example device as disclosed herein according to one implementation.

FIG. 34. Schematic view of an example device as disclosed herein according to one implementation.

FIG. 35. Schematic view of an example device as disclosed herein according to one implementation.

FIG. 36. Schematic view of an example device as disclosed herein according to one implementation.

FIG. 37. Schematic view of an example device as disclosed herein according to one implementation.

FIG. 38. Schematic view of an example device as disclosed herein according to one implementation.

FIG. 39. Schematic view of an example device as disclosed herein according to one implementation.

FIG. 40. Schematic view of an example device as disclosed herein according to one implementation.

FIG. 41. Schematic view of an example device as disclosed herein according to one implementation.

FIG. 42. Schematic view of an example device as disclosed herein according to one implementation.

FIG. 43. Schematic view of an example device as disclosed herein according to one implementation.

FIG. 44. Photograph of an example device.

FIG. 45. Photograph of an example device.

FIG. 46. Photograph of an example device.

### DETAILED DESCRIPTION

The devices and methods described herein may be understood more readily by reference to the following detailed description of specific aspects of the disclosed subject matter and the examples included therein.

Before the present devices and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal embodiment. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Biocompatible” and “biologically compatible”, as used herein, generally refer to compounds and/or compositions that are, along with any metabolites or degradation products thereof, generally non-toxic to normal cells and tissues, and which do not cause any significant adverse effects to normal cells and tissues when cells and tissues are incubated (e.g., cultured) in their presence.

The term “biodegradable” as used herein refers to a material or substance wherein physical dissolution and/or chemical degradation is effected under physiological conditions.

As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

As used herein, “reduce” or other forms of the word, such as “reducing” or “reduction,” refers to lowering of an event or characteristic (e.g., microbe population/infection). It is understood that the reduction is typically in relation to some standard or expected value. For example, “reducing microbial infection” means reducing the spread of a microbial infection relative to a standard or a control.

As used herein, “prevent” or other forms of the word, such as “preventing” or “prevention,” refers to stopping a particular event or characteristic, stabilizing or delaying the development or progression of a particular event or characteristic, or minimizing the chances that a particular event or characteristic will occur. “Prevent” does not require comparison to a control as it is typically more absolute than, for example, “reduce.” As used herein, something could be reduced but not prevented, but something that is reduced could also be prevented. Likewise, something could be prevented but not reduced, but something that is prevented could also be reduced.

As used herein, “treat” or other forms of the word, such as “treated” or “treatment,” refers to administration of a composition or performing a method in order to reduce, prevent, inhibit, or eliminate a particular characteristic or event (e.g., microbe growth or survival). The term “control” is used synonymously with the term “treat.”

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “breast implant” and “implant”, as used herein, generally refer to medical devices that are implanted either under muscle, under breast tissue, under skin (e.g., breast skin, mastectomy skin, skin graft, etc.), or above the chest muscle for breast augmentation, reconstruction, and/or cosmetic improvement. Such implants can include saline filled or silicone gel implants, or other implants that provide volume for breast augmentation. The terms “breast tissue expander,” “tissue expander,” and “expander,” as used herein, generally refer to devices that are implanted over or under chest muscle, and which are expanded over time to stretch breast skin or other tissue and skin. The tissue expander can be made from any biocompatible and/or non-reactive material commonly used in implantable medical devices, such as standard tissue expanders or breast implants.

As used herein, “molecular weight” refers to the number average molecular weight as measured by ¹H NMR spectroscopy, unless indicated otherwise.

### Devices

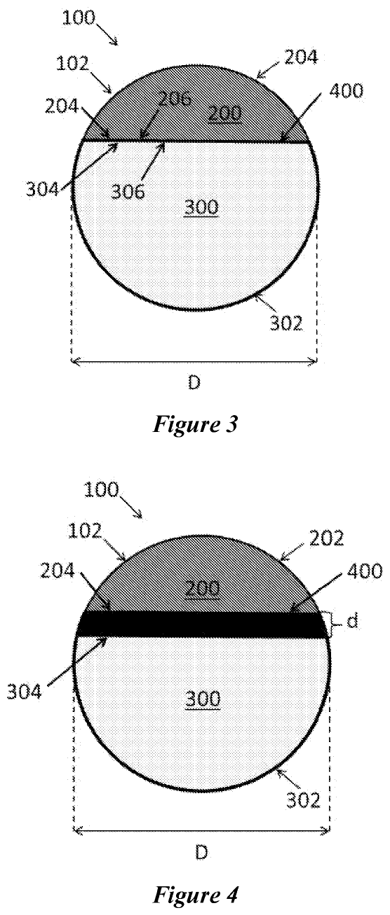

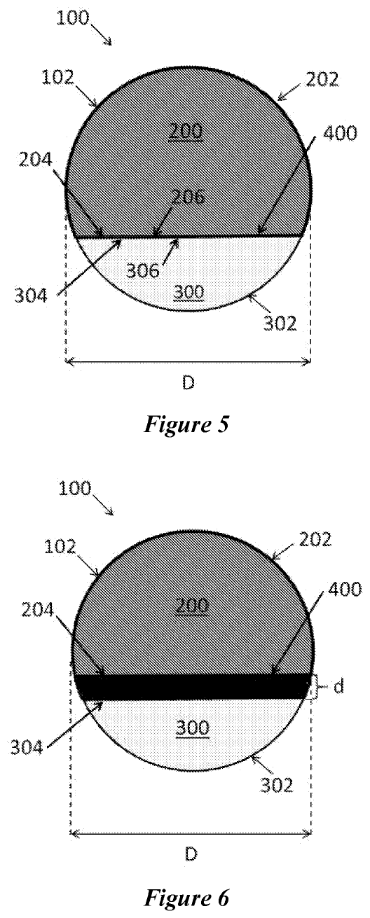

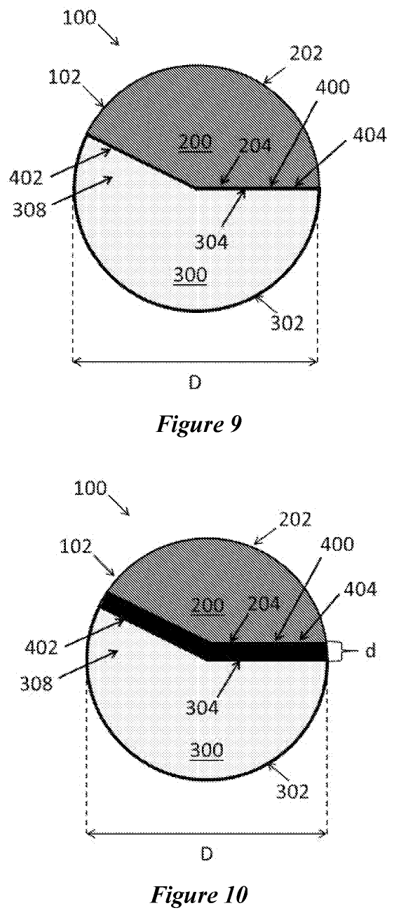

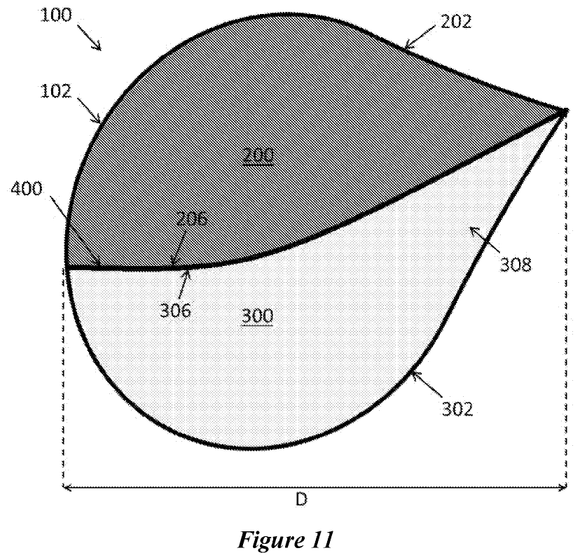

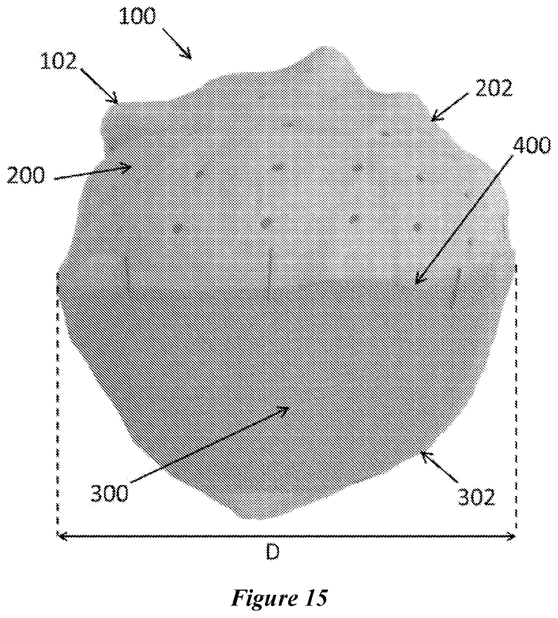

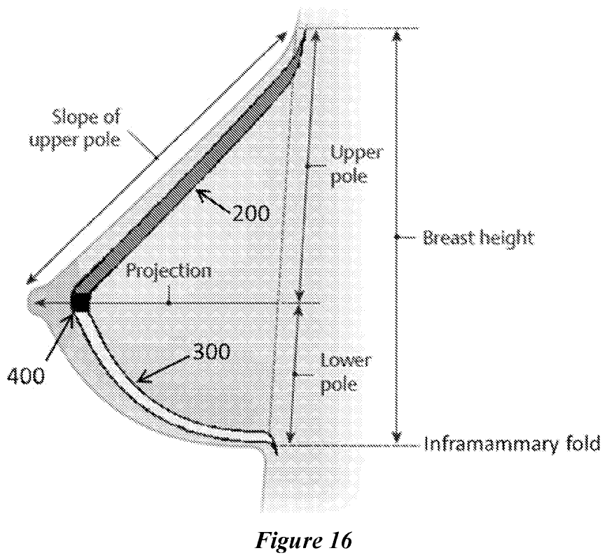

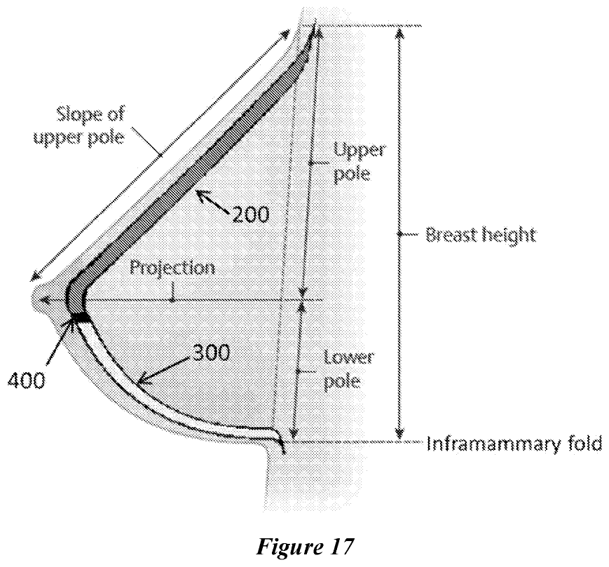

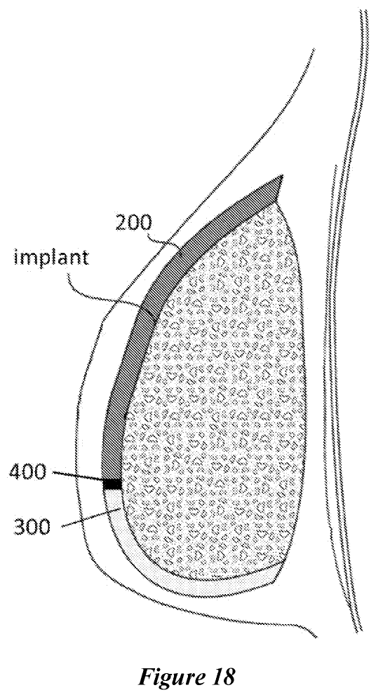

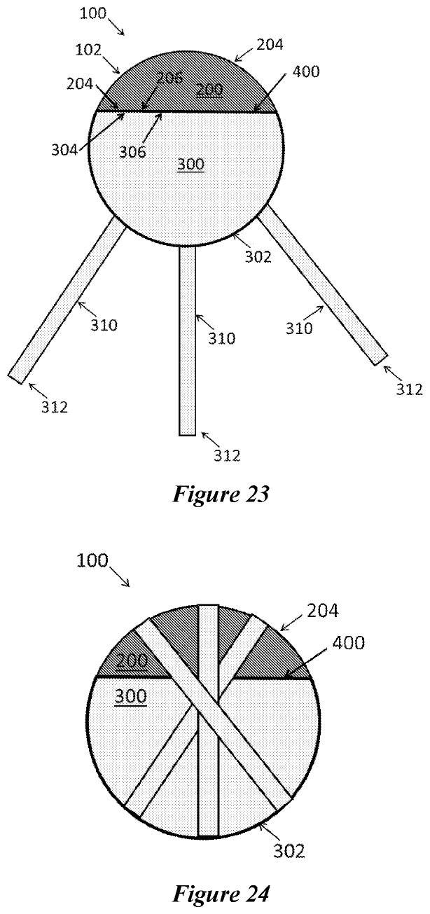

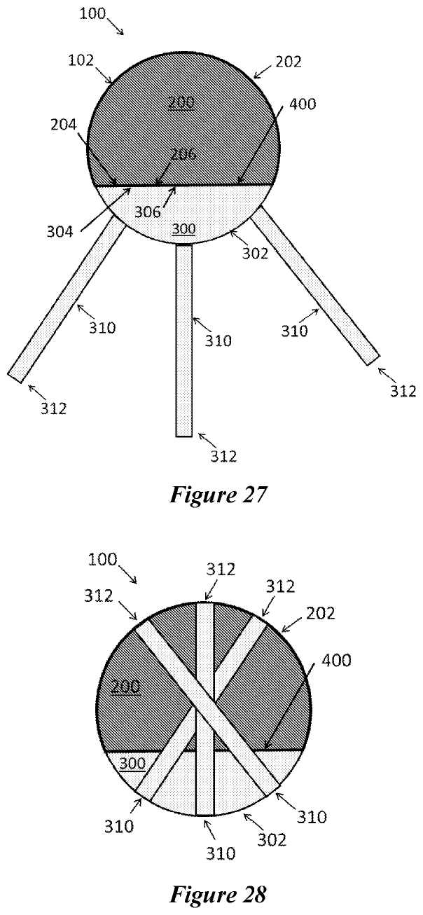

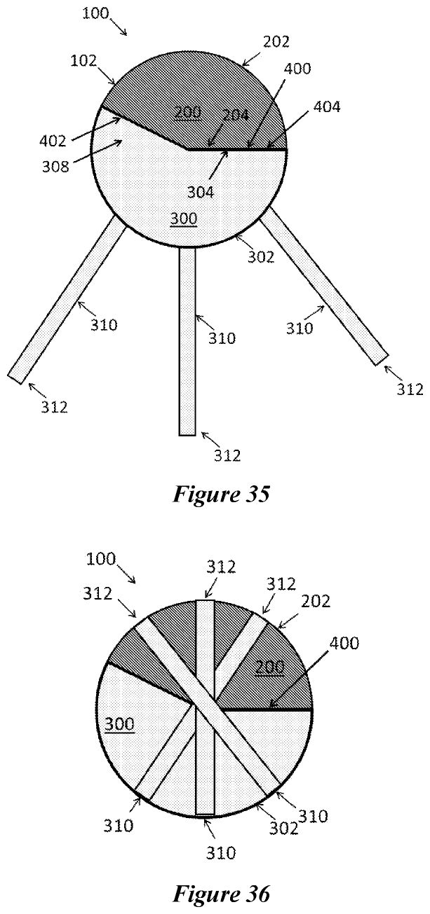

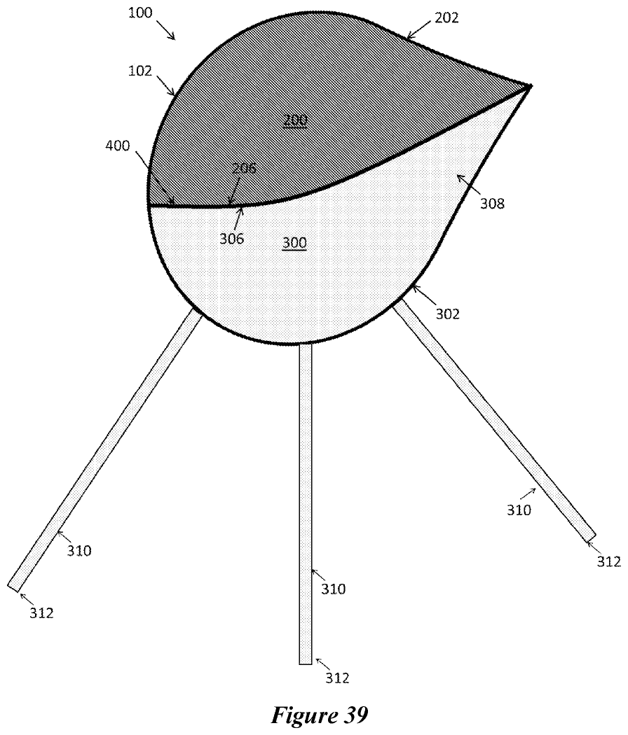

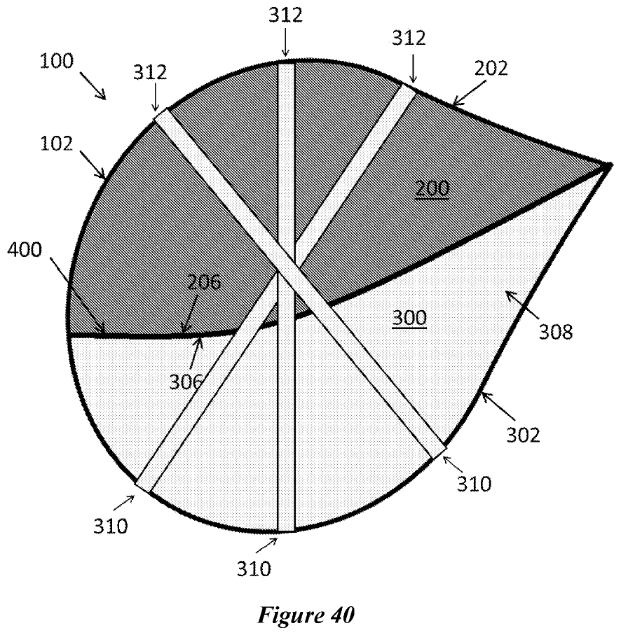

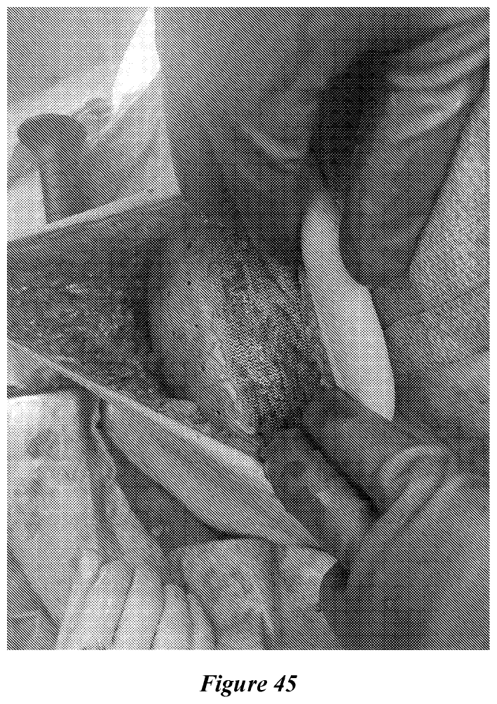

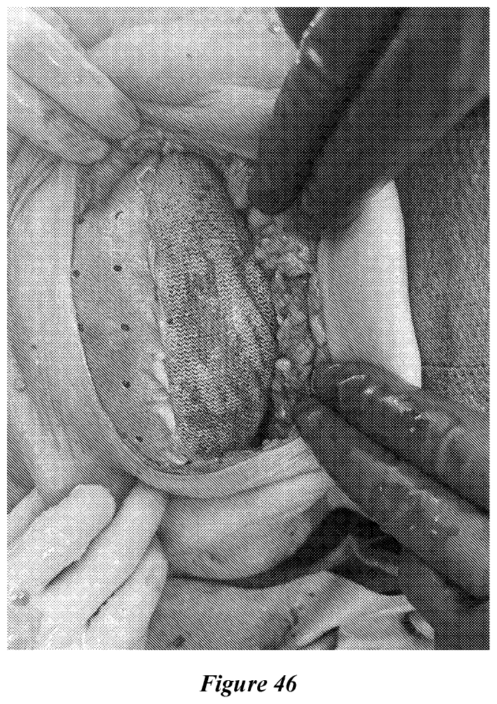

Disclosed herein are devices 100, for example as shown in FIG. 1-FIG. 46. For example, disclosed herein are devices 100 for facilitating reconstruction, augmentation, and/or cosmetic improvement of a breast, the breast having an upper pole, a lower pole, a most projecting portion, and an inframammary fold (e.g., as shown in FIG. 16 and FIG. 17). In some examples, the device 100 is configured to be utilized with a breast implant or tissue expander. In some examples, the device is biocompatible.

The devices 100 comprise a superior portion 200 comprising a biological matrix sheet. The superior portion 200 has a lower end 204 positioned towards the most projecting portion. The superior portion 200 is configured to extend superiorly from the most projecting portion of the breast and form a slope extending along the upper pole of the breast.

The biological matrix sheet can comprise any suitable material. For example, the biological matrix sheet can be selected in view of a variety of factors, such as its thickness, its mechanical properties (e.g., strength, flexibility, stretchability, etc.), its ability to provide soft tissue coverage, its ability to provide a plane for fat grafting, its ability to support upper mastectomy flaps, its ability to cover implant or expander irregularity, its reversibility, or a combination thereof. In some examples, the biological matrix sheet material is selected such that the posterior and anterior surfaces of the superior portion 200 are substantially the same.

In some examples, the biological matrix sheet can comprise any suitable biologic material, such as, for example, autologous or cadaveric tissue, and/or biomatrices, such as, for example, ATM.

As used herein, ATM refers to a tissue-derived biomatrix structure that can be made from any of a wide range of collagen-containing tissues by removing all, or substantially all, viable cells and all detectable subcellular components and/or debris generated by killing cells. As used herein, an ATM lacking “substantially all viable cells” is an ATM in which the concentration of viable cells is less than 1% (e.g., less than: 0.1%; 0.01%; 0.001%; 0.0001%; 0.00001%; or 0.000001%) of that in the tissue or organ from which the ATM was made.

ATM's that are suitable for use in the present disclosure include those that contain, lack, or substantially lack, an epithelial basement membrane. As used herein, an ATM that “substantially lacks” an epithelial basement membrane is an acellular tissue matrix containing less than 5% (e.g., less than: 3%; 2%; 1%; 0.5%; 0.25%; 0.1%; 0.01%; 0.001%; or even less than 0.0001%) of the epithelial basement membrane possessed by the corresponding unprocessed tissue from which the acellular tissue matrix was derived.

An epithelial basement membrane is a thin sheet of extracellular material contiguous with the basilar aspect of epithelial cells. Sheets of aggregated epithelial cells form an epithelium. Thus, for example, the epithelium of skin is called the epidermis, and the skin epithelial basement membrane lies between the epidermis and the dermis. The epithelial basement membrane is a specialized extracellular matrix that provides a barrier function and an attachment surface for epithelial-like cells; however, it does not contribute any significant structural or biomechanical role to the underlying tissue (e.g., dermis). Components of epithelial basement membranes include, for example, laminin, collagen type VII, and nidogen. The temporal and spatial organizations of the epithelial basement membrane distinguish it from, e.g., the dermal extracellular matrix.

Accordingly, in some examples, the ATMs suitable for use in the present disclosure contain epithelial basement membrane. In other examples, ATM may lack or substantially lack epithelial basement membrane.

ATM's suitable for use in the present disclosure may, for example, retain certain biological functions, such as cell recognition, cell binding, the ability to support cell spreading, cell proliferation, cellular in-growth and cell differentiation. Such functions may be provided, for example, by undenatured collagenous proteins (e.g., type I collagen) and a variety of non-collagenous molecules (e.g., proteins that serve as ligands for either molecules such as integrin receptors, molecules with high charge density such as glycosaminoglycans (e.g., hyaluronan) or proteoglycans, or other adhesins). In some examples, the ATM's may retain certain structural functions, including maintenance of histological architecture and maintenance of the three-dimensional array of the tissue's components. The ATM's described herein may also, for example, exhibit desirable physical characteristics such as strength, elasticity, and durability, defined porosity, and retention of macromolecules.

ATMs suitable for use in the present disclosure may be crosslinked or uncrosslinked.

The efficiency of the biological functions of an ATM can be measured, for example, by the ability of the ATM to support cell proliferation. In some examples of the present disclosure, the ATM exhibits at least 50% (e.g., at least: 50%; 60%; 70%; 80%; 90%; 95%; 98%; 99%; 99.5%; 100%; or more than 100%) of that of the native tissue or organ from which the ATM is made.

In some examples, the graft material is amenable to being remodeled by infiltrating cells such as differentiated cells of the relevant host tissue, stem cells such as mesenchymal stem cells, or progenitor cells. This may be accomplished, for example, by forming the grafted matrix material from tissue that is identical to the surrounding host tissue, but such identity is not necessary.

Remodeling may be directed by the above-described ATM components and signals from the surrounding host tissue (such as cytokines, extracellular matrix components, biomechanical stimuli, and bioelectrical stimuli). For example, the presence of mesenchymal stem cells in the bone marrow and the peripheral circulation has been documented in the literature and shown to regenerate a variety of musculoskeletal tissues [Caplan (1991) J. Orthop. Res. 9: 641-650; Caplan (1994) Clin. Plast. Surg. 21: 429-435; and Caplan et al. (1997) Clin Orthop. 342: 254-269]. Additionally, the graft can provide some degree (greater than threshold) of tensile and biomechanical strength during the remodeling process.

ATM in accordance with the present disclosure may be manufactured from a variety of source tissues. For example, ATM may be produced from any collagen-containing soft tissue and muscular skeleton (e.g., dermis, fascia, pericardium, dura, umbilical cords, placentae, cardiac valves, ligaments, tendons, vascular tissue (arteries and veins such as saphenous veins), neural connective tissue, urinary bladder tissue, ureter tissue, or intestinal tissue), as long as the above-described properties are retained by the matrix.

While an ATM may be made from one or more individuals of the same species as the recipient of the ATM graft, this is not necessarily the case. Thus, for example, an ATM may be made from porcine tissue and implanted in a human patient. Species that can serve as recipients of ATM and donors of tissues or organs for the production of the ATM include, without limitation, humans, nonhuman primates (e.g., monkeys, baboons, or chimpanzees), porcine, bovine, horses, goats, sheep, dogs, cats, rabbits, guinea pigs, gerbils, hamsters, rats, or mice.

In some examples, the biological matrix sheet comprises a acellular tissue matrix (ATM), such as an acellular dermal matrix (ADM). In some examples, the biological matrix sheet is derived from human dermis, animal dermis, or animal material.

In some examples, the biological matrix sheet comprises FlexHD pliable ADM, FlexHD pliable MAX ADM, AlloDerm®, DermACELL (e.g., DermACELL AWM®), etc.), porcine ADM, STRATTICE®, CORTIVA™ (e.g., CORTIVA™ allograft dermis), ALLOMAX™, SurgiMend®, ARTIA™, or a combination thereof.

For example, suitable tissue matrices for the biological matrix sheet include ALLODERM®) and STRATTICE®, which are human and porcine acellular dermal matrices produced by LIFECELL® CORPORATION. Alternatively, other suitable acellular tissue matrices can be used as the biological matrix sheet. For example, a number of biological scaffold materials as described by Badylak et al., or any other similar materials, can be used to produce tissues with a stable three-dimensional shape (Badylak et al., “Extracellular Matrix as a Biological Scaffold Material: Structure and Function,” Acta Biomaterialia (2008), doi:10.1016/j.actbio.2008.09.013). Any FDA approved biological tissue product can be used as the biological matrix sheet.

The superior portion 200 can be a layer or any other suitable configuration for providing sufficient support to a breast implant or tissue expander, such as multiple layers, a mesh, or a pad. In some examples, the superior portion 200 is perforated (e.g., by a plurality of perforations).

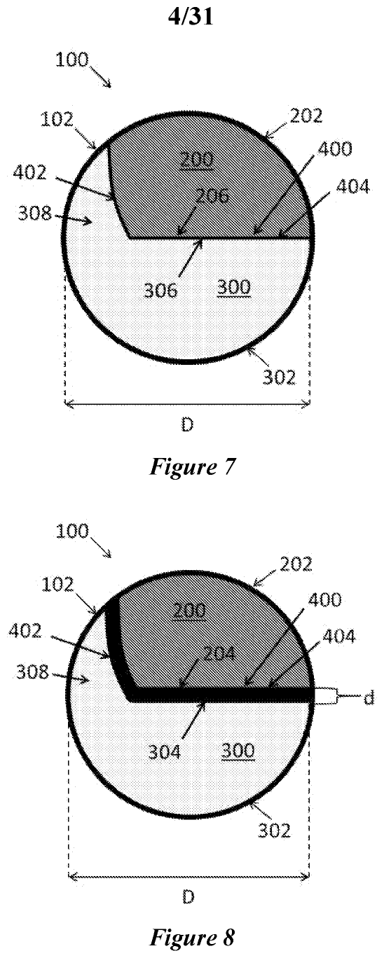

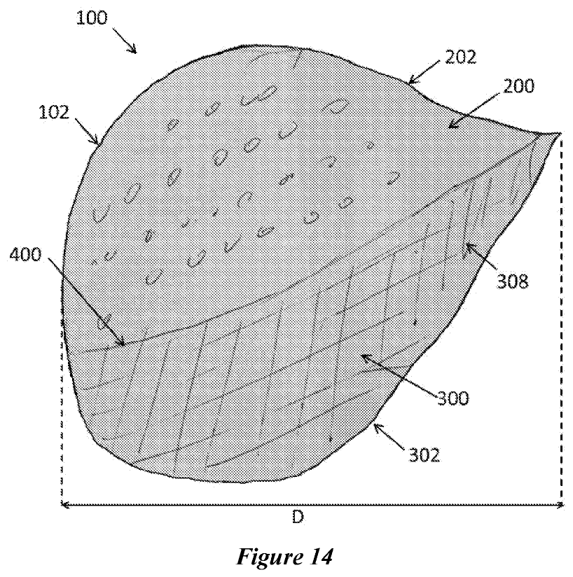

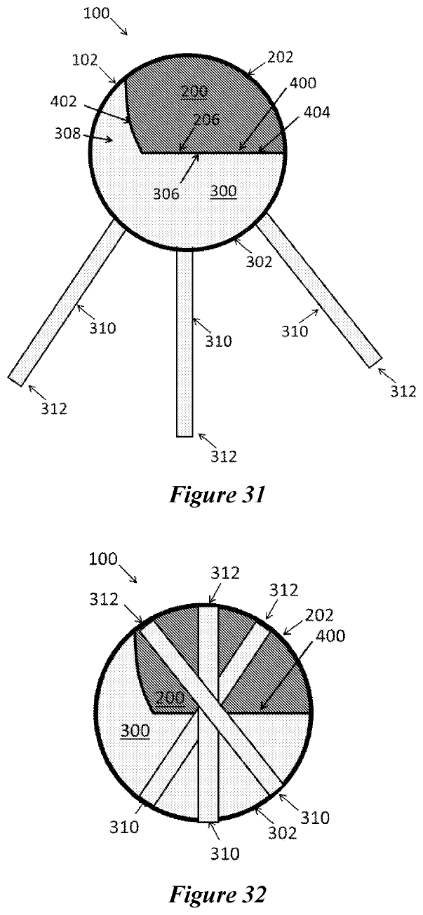

The devices 100 further comprise an inferior portion 300 comprising a synthetic mesh sheet. The inferior portion 300 has an upper end 304 positioned towards the most projecting portion. The inferior portion 300 is configured to extend inferiorly from the upper end 304 and form an inward curvature extending along the lower pole of the breast towards the inframammary fold. In some examples, the inferior portion 300 further includes a lateral portion 308 extending at least partially into the upper pole (e.g., as shown in FIG. 7-FIG. 12, FIG. 14, and FIG. 31-FIG. 42). In use, the lateral portion can help provide a more aesthetic shape to the breast, for example by pushing the breast implant or expander towards the center and providing more cleavage.

The synthetic mesh sheet can comprise any suitable material. In some examples, the synthetic mesh sheet is biodegradable. In some examples, the synthetic mesh sheet is derived from a polymer, such as a biocompatible and/or biodegradable polymer.

In some examples, the synthetic mesh sheet comprises vicryl mesh, gortex, poly(4-Hydroxybuterate) (e.g., Galaflex), Polydioxanone (PDO) (e.g., Durasorb), polyglycolic acid trimethylene carbonate (PGA-PMC) (e.g., TIGR matrix), silk (e.g., SERI scaffold), polypropylene and/or polyglycolic acid (e.g., OviTex®), or a combination thereof. In some examples, the synthetic mesh sheet is formed of polyglycolic acid. In some examples, the synthetic mesh sheet is formed of polyglactin 910. In some examples, the synthetic mesh sheet is formed of Vicryl®, produced by ETHICON® CORPORATION.

For example, the synthetic mesh sheet can be selected in view of a variety of factors, such as its mechanical properties, such as its strength.

In some examples, the synthetic mesh sheet has a strength profile of 15 Newtons per centimeter (N/cm) or more (e.g., 16 N/cm or more, 17 N/cm or more, 18 N/cm or more, 19 N/cm or more, 20 N/cm or more, 25 N/cm or more, 30 N/cm or more, 35 N/cm or more, 40 N/cm or more, 45 N/cm or more, 50 N/cm or more, 55 N/cm or more, 60 N/cm or more, 65 N/cm or more, 70 N/cm or more, 75 N/cm or more, 80 N/cm or more, 85 N/cm or more, 90 N/cm or more, 95 N/cm or more, 100 N/cm or more, 110 N/cm or more, 120 N/cm or more, 130 N/cm or more, or 140 N/cm or more). In some examples, the synthetic mesh sheet has a strength profile of 150 N/cm or less (e.g., 140 N/cm or less, 130 N/cm or less, 120 N/cm or less, 110 N/cm or less, 100 N/cm or less, 95 N/cm or less, 90 N/cm or less, 85 N/cm or less, 80 N/cm or less, 75 N/cm or less, 70 N/cm or less, 65 N/cm or less, 60 N/cm or less, 55 N/cm or less, 50 N/cm or less, 45 N/cm or less, 40 N/cm or less, 35 N/cm or less, 30 N/cm or less, 25 N/cm or less, 20 N/cm or less, 19 N/cm or less, 18 N/cm or less, 17 N/cm or less, or 16 N/cm or less). The strength profile of the synthetic mesh can range from any of the minimum values described above to any of the maximum values described above. For example, the synthetic mesh can have a strength profile of from 15 Newtons per centimeter (N/cm) to 150 N/cm (e.g., from 15 to 80 N/cm, from 80 to 150 N/cm, from 15 to 50 N/cm, from 50 to 100 N/cm, from 100 to 150 N/cm, from 15 to 125 N/cm, from 15 to 100 N/cm, from 15 to 75 N/cm, from 15 to 25 N/cm, from 25 to 150 N/cm, from 50 to 150 N/cm, from 75 to 150 N/cm, from 125 to 150 N/cm, from 20 to 140 N/cm, or from 25 to 125 N/cm).

In some examples, the synthetic mesh sheet has an initial burst strength of 50 Newtons (N) or more (e.g., 55 N or more, 60 N or more, 65 N or more, 70 N or more, 75 N or more, 80 N or more, 85 N or more, 90 N or more, 95 N or more, 100 N or more, 110 N or more, 120 N or more, 130 N or more, 140 N or more, 150 N or more, 160 N or more, 170 N or more, 180 N or more, 190 N or more, 200 N or more, 225 N or more, 250 N or more, 275 N or more, 300 N or more, or 325 N or more). In some examples, the synthetic mesh sheet has an initial burst strength of 350 N or less (e.g., 325 N or less, 300 N or less, 275 N or less, 250 N or less, 225 N or less, 200 N or less, 190 N or less, 180 N or less, 170 N or less, 160 N or less, 150 N or less, 140 N or less, 130 N or less, 120 N or less, 110 N or less, 100 N or less, 95 N or less, 90 N or less, 85 N or less, 80 N or less, 75 N or less, 70 N or less, 65 N or less, 60 N or less, or 55 N or less). The initial burst strength of the synthetic mesh can range from any of the minimum values described above to any of the maximum values described above. For example, the synthetic mesh can have an initial burst strength of from 50 Newtons (N) to 350 N (e.g., from 50 to 200 N, from 200 to 250 N, from 50 to 150 N, from 150 to 250 N, from 250 to 350 N, from 50 to 100 N, from 100 to 150 N, from 150 to 200 N, from 200 to 250 N, from 250 to 300 N, from 300 to 150 N, from 50 to 325 N, from 50 to 300 N, from 50 to 275 N, from 50 to 250 N, from 50 to 225 N, from 50 to 175 N, from 50 to 125 N, from 50 to 75 N, from 75 to 350 N, from 100 to 350 N, from 125 to 350 N, from 150 to 350 N, from 175 to 350 N, from 225 to 350 N, from 275 to 350 N, from 325 to 350 N, from 75 to 325 N, or from 100 to 300 N).

The devices 100 further comprise a connection portion 400 configured to connect the lower end 204 of the superior portion to the upper end 304 of the inferior portion. In some examples, at least a portion of the connection portion 400 is positioned at or below the most projecting portion of the breast. In some examples, the connection portion 400 is a seam extending between the lower end 204 and the upper end 304. In some examples, the superior portion 200 is contiguous with the inferior portion 300 along the connection portion 400.

In some examples, the lower end 204 includes a lower edge 206, the upper end 304 includes an upper edge 306, and the lower edge 206 is connected to the upper edge 306 to form at least a portion of the connection portion 400.

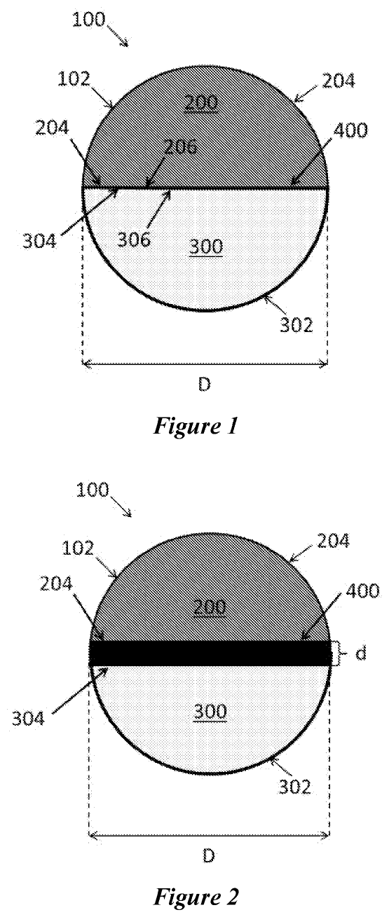

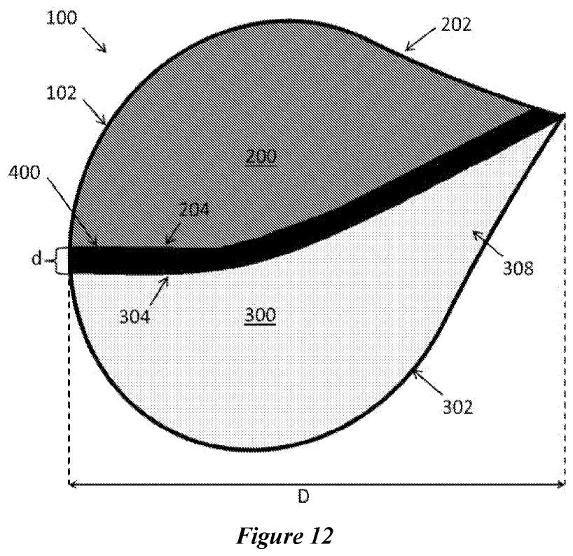

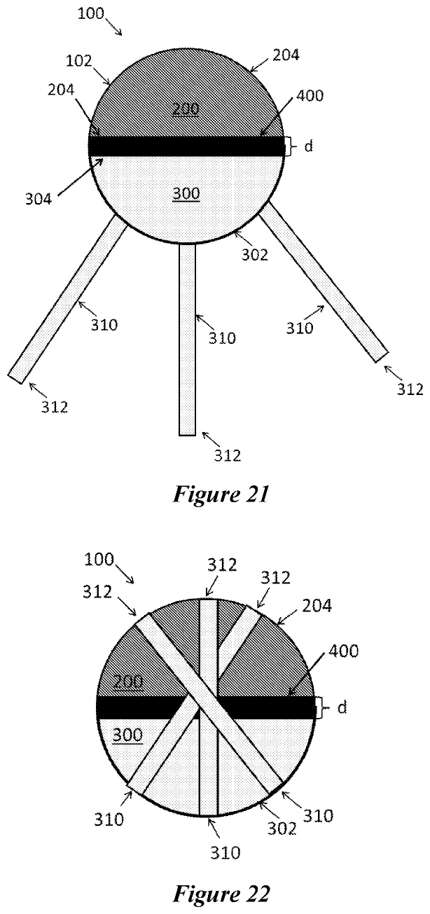

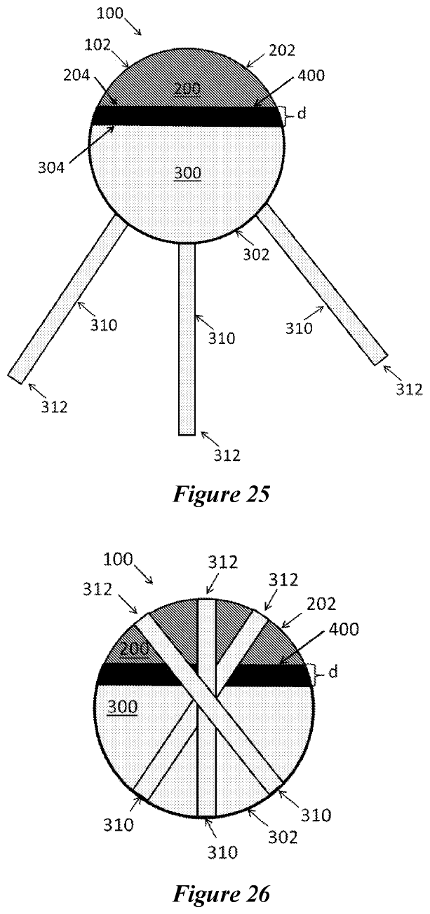

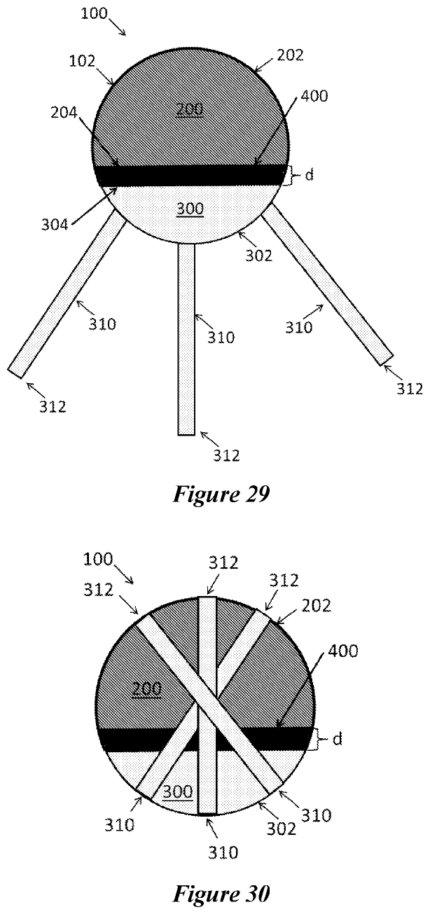

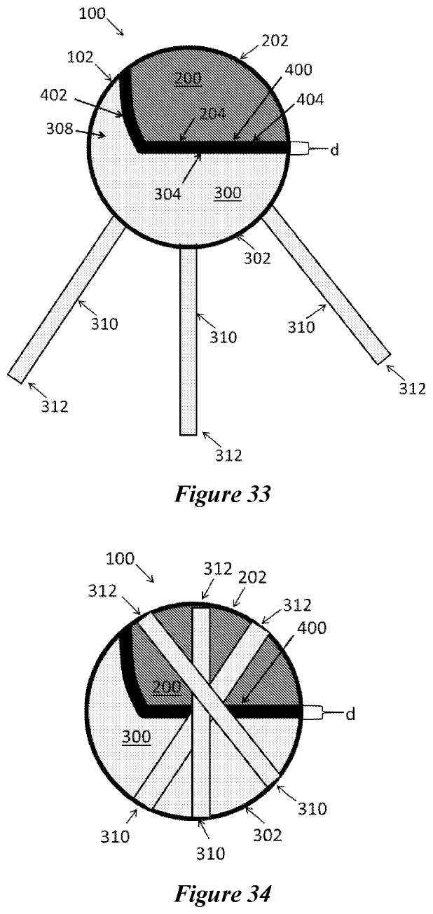

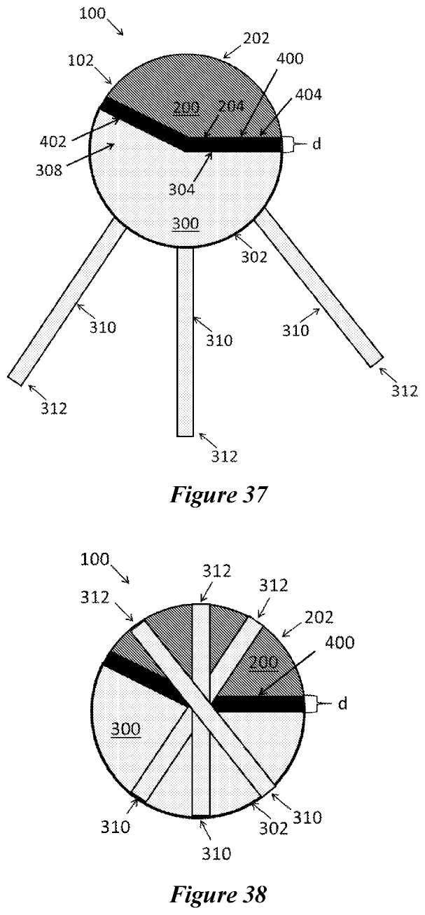

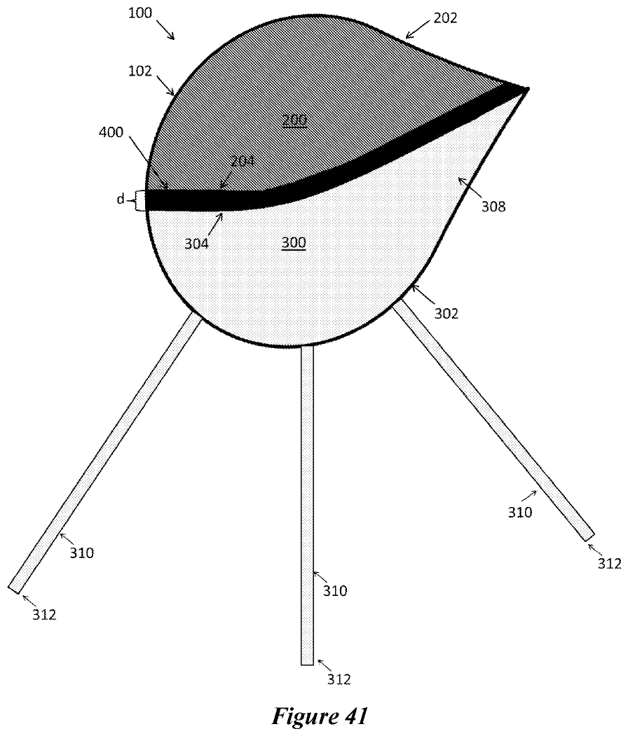

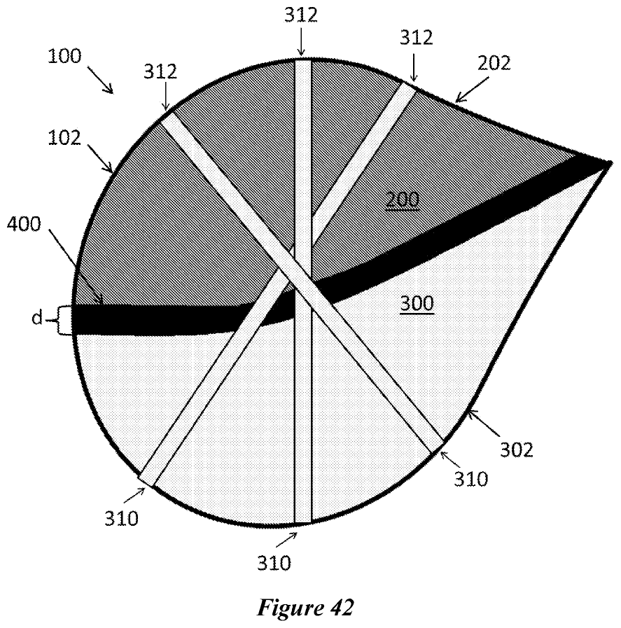

In some examples, the superior portion 200 and the inferior portion 300 overlap along the connection portion 400. In some examples, the superior portion 200 and the inferior portion 300 overlap by a distance (d) along the connection portion 400 (e.g., as shown in FIG. 2, FIG. 4, FIG. 6, FIG. 8, FIG. 10, FIG. 12, FIG. 21, FIG. 22, FIG. 25, FIG. 26, FIG. 29, FIG. 30, FIG. 33, FIG. 34, FIG. 37, FIG. 38, FIG. 41, and FIG. 42).

In some examples, the superior portion 200 and the inferior portion 300 overlap by a distance (d) of 0.1 centimeters (cm) or more along the connection portion 400 (e.g., 0.2 cm or more, 0.3 cm or more, 0.4 cm or more, 0.5 cm or more, 0.6 cm or more, 0.7 cm or more, 0.8 cm or more, 0.9 cm or more, 1 cm or more, 1.25 cm or more, 1.5 cm or more, 1.75 cm or more, 2 cm or more, 2.25 cm or more, 2.5 cm or more, 3 cm or more, 3.5 cm or more, 4 cm or more, 4.5 cm or more, 5 cm or more, 5.5 cm or more, 6 cm or more, 6.5 cm or more, 7 cm or more, 7.5 cm or more, 8 cm or more, 8.5 cm or more, or 9 cm or more). In some examples, the superior portion 200 and the inferior portion 300 overlap by a distance (d) of 10 cm or less along the connection portion 400 (e.g., 9.5 cm or less, 9 cm or less, 8.5 cm or less, 8 cm or less, 7.5 cm or less, 7 cm or less, 6.5 cm or less, 6 cm or less, 5.5 cm or less, 5 cm or less, 4.5 cm or less, 4 cm or less, 3.5 cm or less, 3 cm or less, 2.5 cm or less, 2.25 cm or less, 2 cm or less, 1.75 cm or less, 1.5 cm or less, 1.25 cm or less, 1 cm or less, 0.9 cm or less, 0.8 cm or less, 0.7 cm or less, 0.6 cm or less, 0.5 cm or less, 0.4 cm or less, or 0.3 cm or less). The distance (d) that the superior portion 200 and the inferior portion 300 overlap along the connection portion 400 can range from any of the minimum values described above to any of the maximum values described above. For example, the superior portion 200 and the inferior portion 300 overlap by a distance (d) of from 0.1 centimeter (cm) to 10 cm (e.g., from 0.1 to 5 cm, from 5 to 10 cm, from 0.1 to 2 cm, from 2 to 4 cm, from 4 to 6 cm, from 6 to 8 cm, from 8 to 10 cm, from 0.1 to 9 cm, from 0.1 to 8 cm, from 0.1 to 7 cm, from 0.1 to 6 cm, from 0.1 to 4 cm, from 0.1 to 3 cm, from 0.1 to 1 cm, from 0.1 to 0.5 cm, from 0.25 to 10 cm, from 0.5 to 10 cm, from 1 to 10 cm, from 2 to 10 cm, from 3 to 10 cm, from 4 to 10 cm, from 6 to 10 cm, from 7 to 10 cm, from 9 to 10 cm, from 0.25 to 9.5 cm, from 0.5 to 9 cm, or from 1 to 8 cm).

The connection portion 400 can have any suitable shape. In some examples, the connection portion 400 is straight, curved, or a combination thereof. In some examples, the connection portion 400 comprises a first section 402 and a second section 404, wherein the first section 402 and the second section 404 each independently is straight or curved (e.g., as shown in FIG. 7-FIG. 10 and FIG. 31-FIG. 38).

The devices 100 the lower end 204 of the superior portion can be connected to the upper end 304 of the inferior portion using any suitable means. For example, the superior portion 200 can be connected to the inferior portion 300 by sutures, staples, glue, weaving, or a combination thereof. In some examples, the superior portion 200 is sewn to the inferior portion 300 with a permanent suture. In some examples, the superior portion 200 is sewn to the inferior portion 300 with a biodegradable suture. In some examples, the superior portion 200 is sewn to the inferior portion 300 with a single suture line.

In some examples, the device 100 has a surface area, the superior portion 200 and the inferior portion 300 together comprising 90% or more of the surface area of the device 100 (e.g., 91% or more, 92% or more, 93% or more, 94% or more, 95% or more, 96% or more, 97% or more, 98% or more, or 99% or more). In some examples, the superior portion 200 and the inferior portion 300 together comprise 100% of the surface area of the device 100.

In some examples, 40% or more of the surface area of the device 100 is the superior portion 200 (e.g., 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, or 59% or more). In some examples, 60% or less of the surface area of the device 100 is the superior portion 200 (e.g., 59% or less, 58% or less, 57% or less, 56% or less, 55% or less, 54% or less, 53% or less, 52% or less, 51% or less, 50% or less, 49% or less, 48% or less, 47% or less, 46% or less, 45% or less, 44% or less, 43% or less, 42% or less, or 41% or less). The portion of the surface area of the device 100 comprising the superior portion 200 can range from any of the minimum values described above to any of the maximum values described above. For example, the superior portion 200 can be from 40 to 60% of the surface area of the device 100 (e.g., from 40% to 50%, from 50% to 60%, from 40% to 45%, from 45% to 50%, from 50% to 55%, from 55% to 60%, from 40% to 55%, from 45% to 60%, or from 45% to 55%). The portion of the surface area of the device 100 comprising the superior portion 200 can be selected in view of a variety of factors, such as, for example, the shape and/or size (e.g., volume) of the breast, breast implant, tissue expander.

In some examples, 40% or more of the surface area of the device 100 is the inferior portion 300 (e.g., 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, or 59% or more). In some examples, 60% or less of the surface area of the device 100 is the inferior portion 300 (e.g., 59% or less, 58% or less, 57% or less, 56% or less, 55% or less, 54% or less, 53% or less, 52% or less, 51% or less, 50% or less, 49% or less, 48% or less, 47% or less, 46% or less, 45% or less, 44% or less, 43% or less, 42% or less, or 41% or less). The portion of the surface area of the device 100 comprising the inferior portion 300 can range from any of the minimum values described above to any of the maximum values described above. For example, the inferior portion 300 can be from 40 to 60% of the surface area of the device 100 (e.g., from 40% to 50%, from 50% to 60%, from 40% to 45%, from 45% to 50%, from 50% to 55%, from 55% to 60%, from 40% to 55%, from 45% to 60%, or from 45% to 55%). The portion of the surface area of the device 100 comprising the inferior portion 300 can be selected in view of a variety of factors, such as, for example, the shape and/or size (e.g., volume) of the breast, breast implant, and/or tissue expander.

The device 100 can have any suitable shape. The shape of the device 100 can be selected in view of a variety of factors, such as, for example, the shape and/or size (e.g., volume) of the breast, breast implant, tissue expander. In some examples, the device 100 is symmetric in shape. In some examples, the device 100 is asymmetric in shape. In some examples, the device 100 has a shape that is substantially circular, ovate, ovoid, elliptic, or teardrop.

In some examples, the superior portion 200 has a first edge 202, the inferior portion 300 has a second edge 302, and the device 100 has a periphery 102 comprising at least a portion of the first edge 202 and the second edge 302. In some examples, at least a portion of the first edge 202, the second edge 302, the periphery 102, or a combination thereof is curved.

In some examples, the superior portion 200, the inferior portion 300, the device 100, or a combination thereof is substantially flat. In some examples, the superior portion 200, the inferior portion 300, the device 100, or a combination thereof is shaped (e.g., three dimensionally), such as curved or rounded (e.g., cup shaped).

In some examples, the device 100 is reversible (e.g., posterior and anterior surfaces are substantially the same). For example, the biological matrix sheet material is selected such that the posterior and anterior surfaces of the superior portion 200 are substantially the same, such that the posterior and anterior surfaces of the device 100 are the same, and thus the device is reversible.

The device 100 can have an average characteristic dimension (D, as shown in FIG. 1-FIG. 15). The term “characteristic dimension,” as used herein refers to the largest straight line distance between two points in the plane of the shape of the device 100. “Average characteristic dimension” and “mean characteristic dimension” are used interchangeably herein, and generally refer to the statistical mean characteristic dimension. For example, for a circular device 100, the average characteristic dimension can refer to the average diameter.

In some examples, the device 100 has an average characteristic dimension (e.g., diameter) (D) of 7 centimeters (cm) or more (e.g., 7.5 cm or more, 8 cm or more, 8.5 cm or more, 9 cm or more, 9.5 cm or more, 10 cm or more, 11 cm or more, 12 cm or more, 13 cm or more, 14 cm or more, 15 cm or more, 16 cm or more, 17 cm or more, 18 cm or more, 19 cm or more, 20 cm or more, 21 cm or more, 22 cm or more, 23 cm or more, or 24 cm or more). In some examples, the device 100 has an average characteristic dimension (e.g., diameter) (D) of 25 cm or less (e.g., 24 cm or less, 23 cm or less, 22 cm or less, 21 cm or less, 20 cm or less, 19 cm or less, 18 cm or less, 17 cm or less, 16 cm or less, 15 cm or less, 14 cm or less, 13 cm or less, 12 cm or less, 11 cm or less, 10 cm or less, 9.5 cm or less, 9 cm or less, 8.5 cm or less, or 8 cm or less). The average characteristic dimension (e.g., diameter) (D) of the device 100 can range from any of the minimum values described above to any of the maximum values described above. For example, the device 100 can have an average characteristic dimension (e.g., diameter) (D) of from 7 centimeters to 25 centimeters (e.g., from 7 to 16 cm, from 16 to 25 cm, from 7 to 10 cm, from 10 to 13 cm, from 13 to 16 cm, from 16 to 19 cm, from 19 to 22 cm, from 22 to 25 cm, from 7 to 24 cm, from 7 to 22 cm, from 7 to 20 cm, from 7 to 18 cm, from 7 to 14 cm, from 7 to 12 cm, from 7 to 9 cm, from 8 to 25 cm, from 10 to 25 cm, from 12 to 25 cm, from 14 to 25 cm, from 18 to 25 cm, from 20 to 25 cm, from 23 to 25 cm, from 8 to 24 cm, from 9 to 23 cm, or from 10 to 22 cm).

In some examples, the device 100 is configured to be utilized with a breast implant or tissue expander. In some examples, the device 100 is configured to substantially cover the breast implant or tissue expander. In some examples, the device 100 is provided with or intended to be used with a breast implant or tissue expander. The device 100 can be sold, packaged, or used together or individually, or along with a breast implant or tissue expander.

In some examples, in use, the device is positioned prepectorally or postpectorally, anterior to the breast implant or tissue expander, such that the superior portion 200 of the device 100 is positioned superiorly (towards the upper pole) and the inferior portion 300 of the device 100 is positioned inferiorly (towards the lower pole), and wherein the superior portion 200, the inferior portion 300, or the connection portion 400 of the device is positioned anterior to the most projecting portion of the breast implant or tissue expander. In some examples, in use, the device is positioned prepectorally or postpectorally, anterior to the breast implant or tissue expander, such that the superior portion 200 of the device 100 is positioned anterior to the most projecting portion of the breast implant or tissue expander and superiorly, and the inferior portion 300 of the device 100 is positioned inferiorly, towards the lower pole. In some examples, in use, the device is positioned prepectorally or postpectorally, anterior to the breast implant or tissue expander, such that the superior portion 200 of the device 100 is positioned superiorly (towards the upper pole), and the inferior portion 300 of the device 100 is positioned anterior to the most projecting portion of the breast implant or tissue expander and inferiorly, towards the lower pole.

In some examples, the size and/or shape of the superior portion 200, the inferior portion 300, the device 100, or a combination thereof is selected based on the volume and/or shape of the breast implant or tissue expander. In some examples, the breast implant or tissue expander has an intra-operative fill volume of 0 cubic centimeters (cc) or more (e.g., 5 cc or more, 10 cc or more, 15 cc or more, 20 cc or more, 25 cc or more, 30 cc or more, 35 cc or more, 40 cc or more, 45 cc or more, 50 cc or more, 60 cc or more, 70 cc or more, 80 cc or more, 90 cc or more, 100 cc or more, 125 cc or more, 150 cc or more, 175 cc or more, 200 cc or more, 225 cc or more, 250 cc or more, 275 cc or more, 300 cc or more, 350 cc or more, 400 cc or more, 450 cc or more, 500 cc or more, 550 cc or more, 600 cc or more, 650 cc or more, 700 cc or more, 750 cc or more, 800 cc or more, 850 cc or more, 900 cc or more, 950 cc or more, 1000 cc or more, 1100 cc or more, 1200 cc or more, 1300 cc or more, or 1400 cc or more). In some examples, the breast implant or tissue expander has an intra-operative fill volume of 1500 cc or less (e.g., 1400 cc or less, 1300 cc or less, 1200 cc or less, 1100 cc or less, 1000 cc or less, 950 cc or less, 900 cc or less, 850 cc or less, 800 cc or less, 750 cc or less, 700 cc or less, 650 cc or less, 600 cc or less, 550 cc or less, 500 cc or less, 450 cc or less, 400 cc or less, 350 cc or less, 300 cc or less, 275 cc or less, 250 cc or less, 225 cc or less, 200 cc or less, 175 cc or less, 150 cc or less, 125 cc or less, 100 cc or less, 90 cc or less, 80 cc or less, 70 cc or less, 60 cc or less, 50 cc or less, 45 cc or less, 40 cc or less, 35 cc or less, 30 cc or less, 25 cc or less, 20 cc or less, 15 cc or less, 10 cc or less, or 5 cc or less). The intra-operative fill volume of the breast implant or tissue expander can range from any of the minimum values described above to any of the maximum values described above. For example, the breast implant or tissue expander can have an intra-operative fill volume of from 0 cubic centimeters (cc) to 1500 cc (e.g., from 0 to 750 cc, from 750 to 1500 cc, from 0 to 500 cc, from 500 to 1000 cc, from 1000 to 1500 cc, from 0 to 1400 cc, from 0 to 1200 cc, from 0 to 1000 cc, from 0 to 800 cc, from 0 to 600 cc, from 0 to 400 cc, from 0 to 200 cc, from 0 to 100 cc, from 0 to 50 cc, from 0 to 25 cc, from 25 to 1500 cc, from 50 to 1500 cc, from 100 to 1500 cc, from 200 to 1500 cc, from 400 to 1500 cc, from 600 to 1500 cc, from 800 to 1500 cc, from 1200 to 1500 cc, from 25 to 1400 cc, from 50 to 1300 cc, or from 100 to 1200 cc).

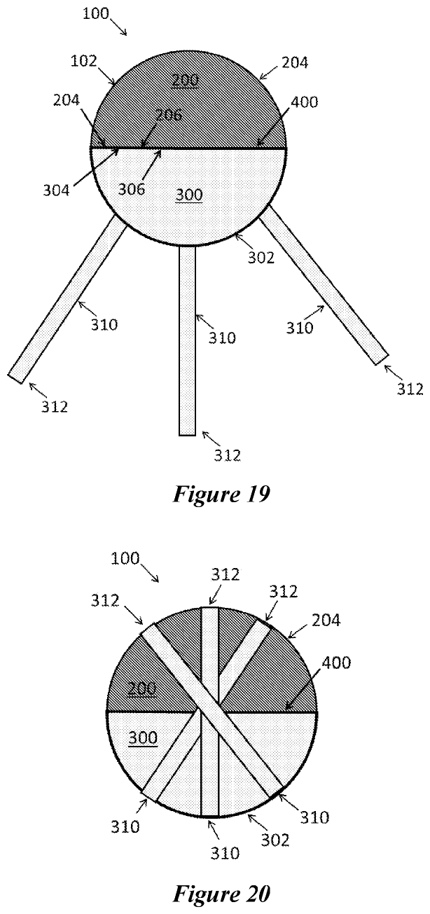

In some examples, the device 100 further comprises one or more (e.g., 2 or more, 3 or more, 4 or more, or 5 or more) strips 310 of synthetic material, each of the one or more strips extending from a portion of the second edge to a distal end 312 (FIG. 19-FIG. 42). In some examples, the distal end 312 of each of the one or more strips 310 is configured to be attached to a portion of the first edge 202, for example such that each of the one of more strips 310 can at least partially encircle the breast, breast implant, and/or tissue expander. Accordingly, in some examples, each of the one or more strips 310 can be configured to help hold the breast, breast implant, and/or tissue expander in place. In some examples, the distal end 312 of each of the one or more strips 310 can be attached to a portion of the first edge 202 by sutures, staples, glue, weaving, or a combination thereof.

In some examples, the device 100 further comprises a therapeutic agent dispersed within the inferior portion 300. In some examples, the therapeutic agent is dispersed inhomogeneously throughout the inferior portion 300 (e.g., randomly, along a concentration gradient, etc.). In some examples, the therapeutic agent is dispersed substantially homogeneously throughout the inferior portion 300.

The therapeutic agent can, for example, comprise an anticancer agent, anti-inflammatory agent, analgesic agent, antimicrobial agent, or a combination thereof. As used herein, antimicrobials include, for example, antibacterials, antifungals, and antivirals.

Examples of antimicrobial agents include, but are not limited to, alexidine, asphodelin A, atromentin, auranthine, austrocortilutein, austrocortirubin, azerizin, chlorbisan, chloroxine, cidex, cinoxacin, citreorosein, copper usnate, cupiennin, curvularin, DBNPA, dehydrocurvularin, desoxyfructo-serotonin, dichloroisocyanuric acid, elaiomycin, holtfreter's solution, malettinin, naphthomycin, neutrolin, niphimycin, nitrocefin, oxadiazoles, paenibacterin, proclin, ritiometan, ritipenem, silicone quaternary amine, stylisin, taurolidine, tirandamycin, trichloroisocyanuric acid, triclocarban, and combinations thereof.

Examples of antibacterials include, but are not limited to, acetoxycycloheximide, aciduliprofundum, actaplanin, actinorhodin, alazopeptin, albomycin, allicin, allistatin, allyl isothiocyanate, ambazone, aminocoumarin, aminoglycosides, 4-aminosalicylic acid, ampicillin, ansamycin, anthramycin, antimycin A, aphidicolin, aplasmomycin, archaeocin, arenicin, arsphenamine, arylomycin A2, ascofuranone, aspergillic acid, avenanthramide, avibactam, azelaic acid, bafilomycin, bambermycin, beauvericin, benzoyl peroxide, blasticidin S, bottromycin, brilacidin, caprazamycin, carbomycin, cathelicidin, cephalosporins, ceragenin, chartreusin, chromomycin A3, citromycin, clindamycin, clofazimine, clofoctol, clorobiocin, coprinol, coumermycin A1, cyclic lipopeptides, cycloheximide, cycloserine, dalfopristin, dapsone, daptomycin, debromomarinone, 17-dimethylaminoethylamino-17-demethoxygeldanamycin, echinomycin, endiandric acid C, enediyne, enviomycin, eravacycline, erythromycin, esperamicin, etamycin, ethambutol, ethionamide, (6S)-6-fluoroshikimic acid, fosfomycin, fosmidomycin, friulimicin, furazolidone, furonazide, fusidic acid, geldanamycin, gentamycin, gepotidacin, glycyclclines, glycyrrhizol, gramicidin S, guanacastepene A, hachimycin, halocyamine, hedamycin, helquinoline, herbimycin, hexamethylenetetramine, hitachimycin, hydramacin-1, isoniazid, kanamycin, katanosin, kedarcidin, kendomycin, kettapeptin, kidamycin, lactivicin, lactocillin, landomycin, landomycinone, lasalocid, lenapenem, leptomycin, lincosamides, linopristin, lipiarmycins, macbecin, macrolides, macromomycin B, maduropeptin, mannopeptimycin glycopeptide, marinone, meclocycline, melafix, methylenomycin A, methylenomycin B, monensin, moromycin, mupirocin, mycosubtilin, myriocin, myxopyronin, naphthomycin A, narasin, neocarzinostatin, neopluramycin, neosalvarsan, neothramycin, netropsin, nifuroxazide, nifurquinazol, nigericin, nitrofural, nitrofurantoin, nocathiacin I, novobiocin, omadacycline, oxacephem, oxazolidinones, penicillins, peptaibol, phytoalexin, plantazolicin, platensimycin, plectasin, pluramycin A, polymixins, polyoxins, pristinamycin, pristinamycin IA, promin, prothionamide, pulvinone, puromycin, pyocyanase, pyocyanin, pyrenocine, questiomycin A, quinolones, quinupristin, ramoplanin, raphanin, resistome, reuterin, rifalazil, rifamycins, ristocetin, roseophilin, salinomycin, salinosporamide A, saptomycin, saquayamycin, seraticin, sideromycin, sodium sulfacetamide, solasulfone, solithromycin, sparassol, spectinomycin, staurosporine, streptazolin, streptogramin, streptogramin B, streptolydigin, streptonigrin, styelin A, sulfonamides, surfactin, surotomycin, tachyplesin, taksta, tanespimycin, telavancin, tetracyclines, thioacetazone, thiocarlide, thiolutin, thiostrepton, tobramycin, trichostatin A, triclosan, trimethoprim, trimethoprim, tunicamycin, tyrocidine, urauchimycin, validamycin, viridicatumtoxin B, vulgamycin, xanthomycin A, xibornol, amikacin, amoxicillin, ampicillin, atovaquone, azithromycin, aztreonam, bacitracin, carbenicillin, cefadroxil, cefazolin, cefdinir, cefditoren, cefepime, cefiderocol, cefoperazone, cefotetan, cefoxitin, cefotaxime, cefpodoxime, cefprozil, ceftaroline, ceftazidime, ceftibuten, ceftizoxime, ceftriaxone, chloramphenicol, colistimethate, cefuroxime, cephalexin, cephradine, cilastatin, cinoxacin, ciprofloxacin, clarithromycin, clindamycin, dalbavancin, dalfopristin, daptomycin, demeclocycline, dicloxacillin, doripenem, doxycycline, eravacycline, ertapenem, erythromycin, fidaxomicin, fosfomycin, gatifloxacin, gemifloxacin, gentamicin, imipenem, lefamulin, lincomycin, linezolid, lomefloxacin, loracarbef, meropenem, metronidazole, minocycline, moxifloxacin, nafcillin, nalidixic acid, neomycin, norfloxacin, ofloxacin, omadacycline, oritavancin, oxacillin, oxytetracycline, paromomy cin, penicillin, pentamidine, piperacillin, plazomicin, quinupristin, rifaximin, sarecycline, secnidazole, sparfloxacin, spectinomycin, sulfamethoxazole, sulfisoxazole, tedizolid, telavancin, telithromycin, ticarcillin, tigecycline, tobramycin, trimethoprim, trovafloxacin, vancomycin, and combinations thereof.

Examples of antifungals include, but are not limited to, abafungin, acibenzolar, acibenzolar-S-methyl, acrisorcin, allicin, aminocandin, amorolfine, amphotericin B, anidulafungin, azoxystrobin, bacillomycin, bacillus pumilus, barium borate, benomyl, binapacryl, boric acid, bromine monochloride, bromochlorosalicylanilide, bupirimate, butenafine, candicidin, caprylic acid, captafol, captan, carbendazim, caspofungin, cerulenin, chloranil, chlormidazole, chlorophetanol, chlorothalonil, chloroxylenol, chromated copper arsenate, ciclopirox, cilofungin, cinnamaldehyde, clioquinol, copper(I) cyanide, copper(II) arsenate, cruentaren, cycloheximide, davicil, dehydroacetic acid, dicarboximide fungicides, dichlofluanid, dimazole, diphenylamine, echinocandin, echinocandin B, epoxiconazole, ethonam, falcarindiol, falcarinol, famoxadone, fenamidone, fenarimol, fenpropimorph, fentin acetate, fenticlor, filipin, fluazinam, fluopicolide, flusilazole, fluxapyroxad, fuberidazole, griseofulvin, halicylindramide, haloprogin, hamycin, hexachlorobenzene, hexachlorocyclohexa-2,5-dien-1-one, 5-hydroxy-2(5H)-furanone, iprodione, lime sulfur, mancozeb, maneb, melafix, metalaxyl, metam sodium, methylisothiazolone, methylparaben, micafungin, miltefosine, monosodium methyl arsenate, mycobacillin, myclobutanil, natamycin, beta-nitrostyrene, nystatin, paclobutrazol, papulacandin B, parietin, pecilocin, pencycuron, pentamidine, pentachloronitrobenzene, pentachlorophenol, perimycin, 2-phenylphenol, polyene antimycotic, propamocarb, propiconazole, pterulone, ptilomycalin A, pyrazophos, pyrimethanil, pyrrolnitrin, selenium disulfide, sparassol, strobilurin, sulbentine, tavaborole, tebuconazole, terbinafine, theonellamide F, thymol, tiabendazole, ticlatone, tolciclate, tolnaftate, triadimefon, triamiphos, tribromometacresol, 2,4,6-tribromophenol, tributyltin oxide, triclocarban, triclosan, tridemorph, trimetrexate, undecylenic acid, validamycin, venturicidin, vinclozolin, vinyldithiin, vusion, xanthene, zinc borate, zinc pyrithione, zineb, ziram, voriconazole, itraconazole, posaconazole, fluconazole, ketoconazole, clotrimazole, isavuconazonium, miconazole, caspofungin, anidulafungin, micafungin, griseofulvin, terbinafine, flucytosine, terbinafine, nystatin, amphotericin b., and combinations thereof.

Examples of antivirals include, but are not limited to, afovirsen, alisporivir, angustific acid, angustifodilactone, alovudine, beclabuvir, 2,3-bis(acetylmercaptomethyl)quinoxaline, brincidofovir, dasabuvir, docosanol, fialuridine, ibacitabine, imiquimod, inosine, inosine pranobex, interferon, metisazone, miltefosine, neokadsuranin, neotripterifordin, ombitasvir, oragen, oseltamivir, pegylated interferon, podophyllotoxin, radalbuvir, semapimod, tecovirimat, telbivudine, theaflavin, tilorone, triptofordin C-2, variecolol, Zmapp, abacavir, acyclovir, adefovir, amantadine, amprenavir, atazanavir, balavir, baloxavir marboxil, boceprevir, cidofovir, cobicistat, daclatasvir, darunavir, delavirdine, didanosine, docasanol, dolutegravir, doravirine, ecoliever, edoxudine, efavirenz, elvitegravir, emtricitabine, enfuvirtide, entecavir, etravirine, famciclovir, fomivirsen, fosamprenavir, forscarnet, fosnonet, famciclovir, favipravir, fomivirsen, foscavir, ganciclovir, ibacitabine, idoxuridine, indinavir, inosine, inosine pranobex, interferon type I, interferon type II, interferon type III, lamivudine, letermovir, letermovir, lopinavir, loviride, maraviroc, methisazone, moroxydine, nelfinavir, nevirapine, nitazoxanide, oseltamivir, peginterferon alfa-2a, peginterferon alfa-2b, penciclovir, peramivir, pleconaril, podophyllotoxin, pyramidine, raltegravir, remdesevir, ribavirin, rilpivirine, rimantadine, rintatolimod, ritonavir, saquinavir, simeprevir, sofosbuvir, stavudine, tarabivirin, telaprevir, telbivudine, tenofovir alafenamide, tenofovir disoproxil, tenofovir, tipranavir, trifluridine, trizivir, tromantadine, umifenovir, valaciclovir, valganciclovir, vidarabine, zalcitabine, zanamivir, zidovudine. And combinations thereof.

In some examples, the therapeutic agent can comprise an anticancer agent. In some examples, the therapeutic agent comprises a chemotherapeutic agent, an immunotherapeutic agent, or a combination thereof.

In some examples, the therapeutic agent can comprise a chemotherapeutic agent. Chemotherapy is the treatment of cancer with one or more cytotoxic anti-neoplastic drugs (e.g., chemotherapeutic agents) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapeutic agents include, but are not limited to, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, LCR, FAM-HYD-1, Marizomib (NPI-0052), Lenalidomide, Carfilzomib, Panobinostat, Quisinostat, Selinexor, Oprozomib, and combinations thereof. The anticancer agent can also include biopharmaceuticals such as, for example, antibodies.

Examples of suitable immunotherapeutic agents include, but are not limited to, alemtuzumab, cetuximab (ERBITUX), gemtuzumab, iodine 131 tositumomab, rituximab, trastuzamab (HERCEPTIN), and combinations thereof.

In some examples, the therapeutic agent can comprise an anti-inflammatory agent, such as steroidal and/or non-steroidal anti-inflammatory agents. Examples of steroidal anti-inflammatory agents include, but are not limited to, hydrocortisone, dexamethasone, prednisolone, prednisone, triamcinolone, methylprednisolone, budesonide, betamethasone, cortisone, and deflazacort. Examples of non-steroidal anti-inflammatory drugs include acetaminophen, aspirin, ibuprofen, naproxen, Celebrex, ketoprofen, tolmetin, etodolac, fenoprofen, flurbiprofen, diclofenac, piroxicam, indomethacin, sulindax, meloxicam, nabumetone, oxaprozin, mefenamic acid, and diflunisal.

In some examples, the therapeutic agent can comprise an analgesic. Examples of analgesics include, but are not limited to, 1-Iodomorphine; 3-Hydroxymorphinan; 4-Methylpregabalin; A-366,833; ABT-202; Aceburic acid; Acefurtiamine; Acetaminosalol; Acetyldihydrocodeine; Acetylmethadol; Adrenorphin; Alazocine; Algifen; Alimadol; Alletorphine; Alphacetylmethadol; Alphamethadol; Amidorphin; Aminophenazone; Ampyrone; Amrutanjan (balm); Anacin; Anadin; Analgecine; Anazocine; Anileridine; Anilopam; Anodyne; Askit Powders; Aspergum; Aspirin; Axomadol; AZD0328; BC Powder; Befiradol; Benorilate; Betacetylmethadol; Betahydroxyfentanyl; Betamethadol; Bicifadine; Biphalin; Brorphine; Bucetin; Bucinnazine; Butalbital; Butinazocine; Butonitazine; Butorphanol; Cannabidiol; Carbazocine; Cebranopadol; Chlorodyne; Chlorproethazine; Cinchophen; Cogazocine; Conolidine; Conorfone; CR-4056; CR665; Dasolampanel; Deltorphin; Deltorphin I; DepoDur; Desmetramadol; Desomorphine; Dezocine; Diacetylnalorphine; Dichloralphenazone; Difenamizole; Dimenoxadol; Dimepheptanol; Dimethylheptylpyran; Dinalbuphine sebacate; Dipipanone; Diproqualone; Dipyrocetyl; Dosulepin; DSP-2230; Embutramide; Enkephalinase nhibitor; Epibatidine; Epiboxidine; Eptazocine; Esterom; Etazocine; Ethylketazocine; Etodesnitazene; Etonitazepipne; Etonitazepyne; Etorphine; Famotidine; Faxeladol; Fedotozine; Fentanyl; Filenadol; Flumexadol; Flumizole; Fluproquazone; Frakefamide; Funapide; Gabapentin; Gabapentin enacarbil; Gabapentinoid; Glafenine; Homofentanyl; Homprenorphine; Ibazocine; Ibuprofen; Incarvillateine; Indantadol; Isomethadone; Isotonitazene; Isovaline; Kavalactone; Kelatorphan; Ketamine; Ketobemidone; Ketorfanol; Ketorolac; Lactucarium; Leconotide; Levallorphan; Levomepromazine; Levomethadone; Lufuradom; Magnesium alicylate; Mavatrep; Meconopsis horridula; Menabitan; Menthol; Menthoxypropanediol; Meprobamate; Meseclazone; Metacetamol; Metamizole; Methoxyflurane; Metkefamide; Metodesnitazene; Metonitazene; Mexiletine; Migraleve; Mirogabalin; Mitragyna speciosa; Moffett's solution; Moramide intermediate; Morpheridine; Morphiceptin; Morphine; Moxazocine; MP-2001; N-2′-IndolyInaltrexamine; Nabilone; Nafoxadol; Nalbuphine; Nalmexone; Naproxen; Nefopam; Nexeridine; NFEPP; Nimesulide; Noracymethadol; Norlevorphanol; Normethadone; Norpipanone; NS-11394; Oliceridine; Opiorphin; Opiranserin; Opium; Otenaproxesul; Oxilorphan; Paracetamol; Pethidine; PF-05089771; Phenacetin; Phenazone; derivatives thereof; and combinations thereof.

In some examples, the therapeutic agent can comprise an analgesic, such as an opioid. Examples of opioids include, but are not limited to, (α/β)-Meprodine; (α/β)-Prodine; 1-(4-Nitrophenylethyl)piperidylidene-2-(4-chlorophenyl)sulfonamide (W-18); 14-Cinnamoyloxycodeinone; 14-Ethoxymetopon; 14-Hydroxydihydrocodeine; 14-Hydroxymorphine; 14-Methoxymetopon; 14-Phenylpropoxymetopon; 18,19-Dehydrobuprenorphine (HS-599); 18-Methoxycoronaridine; 1-Bromocodeine; 1-Chlorocodeine; 1-Iodomorphine Codeine-6-glucuronide; 1-Nitrocodeine; 2,4-Dinitrophenylmorphine; 3-(3-Methoxyphenyl)-3-ethoxycarbonyltropane; 3-(dimethylamino)-2,2-dimethyl-1-phenylpropan-1-one; 3,14-Diacetyloxymorphone; 3,6-Dibutanoylmorphine; 3-Acetyloxymorphone; 3-Allylfentanyl; 3-Hydroxymorphinan; 3-Methylfentanyl; 3-Methylthiofentanyl; 3-Monoacetylmorphine; 4-Chlorophenylpyridomorphinan; 4-Fluoropethidine; 4-Phenylfentanyl; 5,6-Dihydronorsalutaridine; 5,9 alpha-diethy1-2-hydroxybenzomorphan (5,9-DEHB); 6-Acetyldihydromorphine; 6-Keto Nalbuphine; 6-Methyldihydromorphine; 6-Methylenedihydrodesoxymorphine; 6-Monoacetylcodeine; 6-Monoacetylmorphine; 6-Nicotinoyldihydromorphine; 7-Acetoxymitragynine; 7-Hydroxymitragynine; 7-PET; 7-Spiroindanyloxymorphone; 8,14-Dihydroxydihydromorphinone; 8-Carboxamidocyclazocine (8-CAC); Acetorphine; Acetoxyketobemidone; Acetylcodone; Acetyldihydrocodeine; Acetylmethadol; Acetylmorphone; Acetylpropionylmorphine; AD-1211; ADL-5859; AH-7921; Aknadinine; Akuammidine; Akuammine; Alazocine; Alfentanil; Alimadol; Alletorphine (N-allyl-noretorphine); AllyInorpethidine; Allylprodine; Alphaacetylmethadol; Alphamethadol; Alvimopan; Amentoflavone; Anazocine; Anileridine; Anilopam +HCl; Asimadoline; Axomadol; Azaprocin; AZD-2327; Azidomorphine; BDPC; Benzethidine; Benzhydrocodone; Benzylfentanyl; Benzylmorphine; Betacetylmethadol; Betamethadol; Bezitramide; Bisnortilidine; Bremazocine; Brifentanil; BRL-52537; Bromadol; Bromadoline; Bromocodide; Bromoisopropropyldihydromorphinone; Bromomorphide; BU-48; Buprenorphine; Buprenorphine-3-glucuronide; Butinazocine; Butorphanol; Butyrfentanyl; BW373U86; Carbazocine; Carfentanil; Carperidine; Cephakicine; Cephasamine; Chlornaltrexamine; Chlorodihydrocodide; Chloromorphide; Chloroxymorphamine; Ciprefadol; Ciramadol; Clonitazene; Codeine; Codeine methylbromide; Codeine-N-oxide; Codeine-N-oxide (genocodeine); Codeinone; Codide; Codoxime; Cogazocine; Conorfone (codorphone); Coronaridine; Cyclazocine; Cyclorphan; Cyprenorphine; Cyprodime; Cyproterone acetate; Desmethylclozapine; Desmethylmoramide; Desmethylprodine (MPPP); Desocodeine Desomorphine (dihydrodesoxymorphine); Dextromethadone; Dextromoramide; Dextropropoxy phene (propoxy phene); Dezocine; Diacetyldihydromorphine (dihydroheroin, acetylmorphinol); Diampromide; Dibenzoylmorphine; Dibutyrylmorphine; Diethylthiambutene; Difenoxin; Diformylmorphine; Dihydrocodeine; Dihydrocodeine; Dihydrodesoxycodeine (desocodeine); Dihydroetorphine; Dihydroisocodeine; Dihydromorphine; Dimenoxadol; Dimepheptanol (racemethadol); Dimethylmorphine (6-O-Methylcodeine); Dimethylthiambutene; Dioxaphetyl butyrate; Diphenoxylate; Dipipanone; Dipropanoylmorphine; Doxpicomine; DPI-221; DPI-287; DPI-3290; Drotebanol; Droxypropine; Embutramide; Enadoline; Eptazocine; Eseroline; Etazocine; Ethoheptazine; Ethyldihydromorphine; Ethylketazocine; Ethylmethylthiambutene; Ethylmorphine (dionine); Etonitazene; Etorphine; Etoxeridine (carbetidine); Faxeladol; FE 200665; Fedotozine; Fenfangjine G; Fentanyl; Fluorophen; Furethidine; Gemazocine; GR-89696; Herkinorin; Heroin (diacetylmorphine); Heroin-7,8-oxide; Heterocodeine; Hodgkinsine; Homprenorphine; Hydrocodone; Hydromorphinol; Hydromorphone: Hydroxycodeine; Hydroxypethidine (bemidone); HZ-2; Ibazocine; IBNtxA; Ibogaine; IC-26; ICI-199,441; ICI-204,448; Isoaminile; Isocodeine; Isomethadol; Isomethadone; Isotonitazene; Ketamine; Ketazocine; Ketobemidone; Ketorfanol; KNT-42; Kolokol-1; Lefetamine; Levacetylmethadol; Levargorphan; Levoisomethadone; Levomethadone; Levomethorphan; Levomoramide; Levophenacylmorphan; Levopropoxyphene; Levorphanol; Lofentanil; Loperamide; LPK-26; LS-115509; Lufuradom; Matrine; MCOPPB; Menthol; Meperidine-N-oxide; Meptazinol; Metazocine; Metethoheptazine; Methadone; Metheptazine; Methorphan (racemethorphan); Methyldesorphine; Methyldihydromorphine (dihydroheterocodeine); Methyldihydromorphinone; Methylketobemidone; Metofoline; Metonitazene; Metopon; Mirfentanil; Mitragynine; Mitragynine pseudoindoxyl; Morphanol (racemorphanol); Morphenol; Morpheridine; Morphine; Morphine methylbromide; Morphine-6-glucuronide; Morphine-N-oxide; Morphine-N-oxide (genomorphine); Morphinone; Morphol; Moxazocine; MT-45; MT-7716; Myrophine; Nalbuphine; Nalbuphone; Nalfurafine; Nalorphine; Nalorphine dinicotinate; Naltrexol: N-cyclopropylmethylnoretorphine; Nepenthone; Nexeridine; Nicocodeine; Nicodicodeine; Nicomorphine; N-Methylcarfentanil; N-Methylmorphinan; NNC 63-0532; Noracymethadol; Norbuprenorphine; Norbuprenorphine-3-glucuronide; Norcodeine; Noribogaine; Norlevorphanol; Normethadone; Normorphine; Noroxymorphone; Norpipanone; Norpropoxyphene; Nortilidine; N-Phenethyl-14-ethoxymetopon; N-Phenethyl-14-ethoxymetopon; N-Phenethylnordesomorphine; N-Phenethylnormorphine; Ocfentanil; O-Desmethyltramadol; Ohmefentanyl; Opium; Oripavine; Oxilorphan; Oxpheneridine (carbamethidine); Oxycodone; Oxymorphazone; Oxymorphol; Oxymorphone; Pantopon; Papaveretum (Omnopon); Parafluorofentanyl; Pentamorphone; Pentazocine; PEPAP; Pericine: Pethidine (meperidine); Phenadone; Phenadoxone (heptazone); Phenampromide; Phenaridine; Phenazocine; Phencyclidine; Pheneridine; Phenomorphan; Phenoperidine; Pholcodine (morpholinylethylmorphine); Picenadol; Piminodine; Piperidylthiambutene; Piritramide; Prodilidine; Profadol; Proglumide; Proheptazine; Properidine (ipropethidine); Propiram; Propylketobemidone; Prosidol; Proxorphan; Pseudoakuammigine; Pseudomorphine; Pyrrolidinylthiambutene; Pyrroliphene; PZM21; Quadazocine; R-30490; R-4066; Racemoramide; RAM-378; Remifentanil; Ro-1539; Ro4-1539; Ro64-6198; Ro65-6570; RWJ-394,674; Salvinorin A; Salvinorin B ethoxymethyl ether; Salvinorin B methoxymethyl ether; Sameridine; SB-612,111; SC-17599; Semorphone; SKF-10047; SNC-80; SoRI-9409; Spiradoline; SR-16435; SR-8993; Sufentanil; TAN-67; Tannagine; Tapentadol; Tetrapon; Thebacon; Thebacon (acetyldihydrocodeinone, dihydrocodeinone enol acetate); Thebaine; Thenylfentanyl; Thevinone; Thiambutene; Thiazocine; Thienorphine; Thiobromadol (C-8813); Thiofentanyl; Tifluadom; Tilidine; Tonazocine; Tramadol; Transisocodeine; Trefentanil; Trimebutine; Trimeperidine (promedol); U-47700; U-50,488; U-69,593; Viminol; Volazocine; Zenazocine; α-Chlorocodide; α-Chloromorphide; α-hydrocodol; α-Methylacetylfentanyl; α-Methylfentanyl; α-Methylthiofentanyl; β-Chlorocodide; β-hydroxy fentanyl; β-hydroxythiofentanyl; β-Methylfentanyl; ψ-Akuammigine; derivatives thereof; and combinations thereof.

In some examples, the device 100 is implantable in a subject. In some examples, the device 100 is anatomically designed for the subject.

In some examples, at least a portion of the device 100 is biodegradable. In some examples, at least a portion of the device 100 is permanent.

In some examples, the inferior portion 300 is biodegradable. In some examples, the inferior portion 300 is permanent.

In some examples, the inferior portion 300 is configured to be stable for an amount of time after the device is implanted in the subject. As used herein, “stable” means that 10 wt % or less (e.g., 9% or less, 8% or less, 7% or less, 6% or less, 5% or less, 4% or less, 3% or less, 2% or less, or 1% or less) of the inferior portion 300 biodegrades over the selected time period after the device is implanted in the subject.

In some examples, the inferior portion 300 is configured to be stable for an amount of time of 1 month or more after the device 100 is implanted in the subject (e.g., 2 months or more, 3 months or more, 4 months or more, 5 months or more, 6 months or more, 7 months or more, 8 months or more, 9 months or more, 10 months or more, 11 months or more, 1 year or more, 1.25 years or more, 1.5 years or more, 1.75 years or more, 2 years or more, 2.25 years or more, 2.5 years or more, 2.75 years or more, 3 years or more, 3.25 years or more, 3.5 years or more, 3.75 years or more, 4 years or more, 4.25 years or more, or 4.5 years or more). In some examples, the inferior portion 300 is configured to be stable for an amount of time of 5 years or less after the device 100 is implanted in the subject (e.g., 4.75 years or less, 4.5 years or less, 4.25 years or less, 4 years or less, 3.75 years or less, 3.5 years or less, 3.25 years or less, 3 years or less, 2.75 years or less, 2.5 years or less, 2.25 years or less, 2 years or less, 1.75 years or less, 1.5 years or less, 1.25 years or less, 1 year or less, 11 months or less, 10 months or less, 9 months or less, 8 months or less, 7 months or less, 6 months or less, 5 months or less, 4 months or less, 3 months or less, or 2 months or less). The amount of time for which the inferior portion 300 is configured to be stable can range from any of the minimum values described above to any of the maximum values described above. For example, the inferior portion 300 can be configured to be stable for an amount of time of from 1 month to 5 years after the device 100 is implanted in the subject (e.g., from 1 month to 2.5 years, from 2.5 years to 5 years, from 1 month to 1 year, from 1 year to 2 years, from 2 years to 2 years, from 3 years to 4 years, from 4 years to 5 years, from 1 month to 4 years, from 1 month to 3 years, from 1 month to 2 years, from 1 month to 9 months, from 1 month to 6 months, from 1 month to 3 months, from 3 months to 5 years, from 6 months to 5 years, from 9 months to 5 years, from 1 year to 5 years, from 2 years to 5 years, from 3 years to 5 years, from 3 months to 4.5 years, from 6 months to 3.5 years, or from 9 months to 2.5 years).

### Kits

Also disclosed herein are kits comprising any of the devices 100 disclosed herein. For example, the kit can comprise the device 100 disposed within a sterile package.

In some examples, the kit further includes instructions or packaging materials that describe how to administer the device. Containers of the kit can be of any suitable material, e.g., glass, plastic, metal, etc., and of any suitable size, shape, or configuration.

In some examples, the kit can further include a breast implant or tissue expander configured to be used with the device 100. For example, the kit further includes a breast implant or tissue expander configured to be used with the device 100, wherein the device and the breast implant or tissue expander are co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed device and/or product and another component for delivery to a patient.

It is contemplated that the disclosed kits can be used in connection with the disclosed methods of making, the disclosed methods of using, and/or the disclosed devices.

### Methods of Making

Also disclosed herein are methods of manufacturing any of the devices 100 disclosed herein. For example, the methods can comprise connecting the superior portion 200 to the inferior portion 300. In some examples, the superior portion 200 is connected to the inferior portion 300 by sutures, staples, glue, weaving, or a combination thereof. In some examples, the superior portion 200 is sewn to the inferior portion 300 with a permanent suture. In some examples, the superior portion 200 is sewn to the inferior portion 300 with a biodegradable suture. In some examples, the superior portion 200 is sewn to the inferior portion 300 with a single suture line.

In some examples, the methods further comprise making the device based on a 3D model. In some examples, the 3D model is based on an anatomical image of a subject. In some examples, the methods can further comprise collecting the anatomical image of the subject.

### Methods of Use

Also disclosed herein are methods of treating a subject in need thereof, the methods comprising implanting the device into the subject. In some examples, the device is implanted into a breast of the subject. For example, the method can comprise breast reconstruction, augmentation, and/or cosmetic improvement. In some examples, the method can comprise one stage or two stage implant based breast reconstruction. In some examples, the method further comprises anatomically designing the device for the subject.

The device 100 can be implanted subcutaneously within the breast pocket. In some examples, the device can be implanted anterior to pectoral muscle. Although a pre-pectoral position can be preferred in some cases, it should be appreciated that the present devices and methods can be used in a variety of procedures and anatomic locations.

After or during implantation, the device can assist in securing the breast implant or tissue expander in place. For example, the device (e.g., at least a portion of the periphery of the device) can be secured to portions of the chest wall or other appropriate tissue to help position and maintain the breast implant or tissue expander.

Further, the device, positioned anterior to at least a portion of the breast implant or tissue expander, can provide an interface between overlying skin or subcutaneous tissues. Accordingly, the device can provide a number of functions, including supporting the breast implant or tissue expander, providing a material for more rapid or better vascularization, tissue regeneration, and ingrowth, prevention or reduction of scar formation and/or contracture, and/or providing additional tissue to prevent rippling, bottoming out, or any other undesired aesthetic or reconstructive result.

In some examples, the devices described herein can be used to assist in treating patients in whom complications related to breast implants have arisen. Such complications can include malposition (e.g., inframammary fold malposition, lateral malposition, symmastia), stretch deformity, coverage issues (e.g., wrinkling and rippling), and capsular contraction. For example, the devices described herein may be used to help control the breast pocket size and location, act as an “internal bra” to hold the breast implant in place, support fold repairs, support the implant to reduce the pressure and tension on patient's own tissue, and/or provide an additional layer for coverage of the implant.

In some examples, the methods can comprise breast reconstruction and treatment of an oncological disorder, such as breast cancer. In some examples, the devices can further include a therapeutic agent, for example for treatment of the oncological disorder.

For the treatment of oncological disorders, the devices disclosed herein can be administered to a patient in need of treatment in combination with other antitumor or anti-cancer substances and/or with radiation and/or photodynamic therapy and/or with surgical treatment to remove a tumor. These other substances or treatments can be given at the same as or at different times from the devices disclosed herein. For example, the devices disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in °C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Dual Biologic-Synthetic Mesh for Use in One Stage and Two Stage Implant Based Reconstruction

Breast reconstruction is most commonly done with implants. This surgery is usually performed in a two-staged approach (tissue expander—like a water balloon placed at the time of mastectomy first), then a second surgery where the expander is removed and a permanent implant is placed. This can sometimes also be achieved in one stage—with a direct to implant approach (i.e., a permanent implant placed at the time of mastectomy). Regardless of which approach is utilized, plastic surgeons face clinical challenges with implant-based breast reconstruction, including but not limited to: thin mastectomy tissue, physiologic landmarks lost at the time of mastectomy (breast fold), palpability of implant, implant decent, visualized implant rippling, flipping of implant, synmastia. The industry has responded with the use of acellular dermal matrix (ADM) as well as synthetic meshes. Both have benefits, but both have shortcomings due to design and positioning.

Described herein is a dual biologic and synthetic mesh device, wherein the biologic acellular dermal matrix portion is placed in the upper pole (where such coverage is most useful) and the synthetic portion is placed in the lower and lower lateral portion of the breast—that supports the implant and recreates the breast fold and lateral breast. This device is the “best of both worlds” because, historically, the shortcoming of ADM was that overtime the lower pole loses strength and stretches, this is not desirable in the lower breast; secondly, synthetic mesh does not do a good job covering and hiding the upper implant and does not stretch as desired in the most projecting portion of the expander and implant. The devices described herein achieves both in one single device.

The device can be shaped and usable in both direct to implant (DTI) and two-staged expander-based breast reconstruction. The upper pole down to the most projecting portion of the expander is ADM, and the lower and lower lateral portion is a synthetic mesh (e.g., that lasts from 9 months to two or so years).

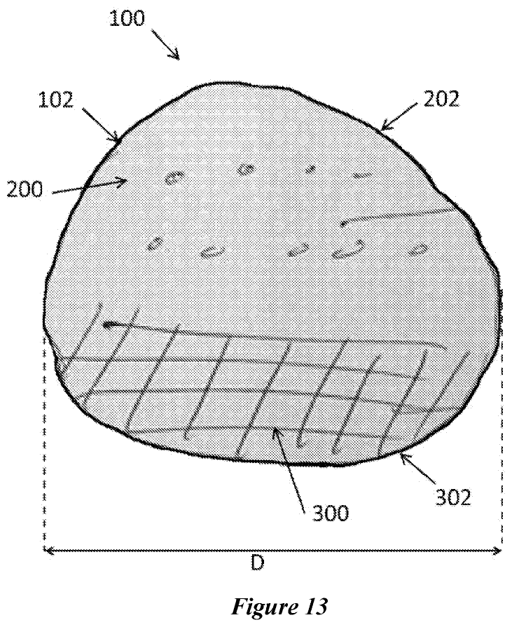

Example shapes of the devices are shown, for example, in FIG. 13-FIG. 15.

The ADM is fully integrated into the synthetic mesh so that when the package is opened a single, shaped sheet of reconstructive biosynthetic mesh is ready for use. In some examples, the ADM cut is deep enough in the cadavers to allow for reversibility—such that there is no sidedness to the ADM (just like there is no sidedness to the mesh)—this provides ease of use and no “right” or “left” device requirements. The device can have the ADM sewn to the synthetic mesh with a permanent suture to keep strength while the expansion process occurs.

The device can be sutured into the chest at the time of tissue expander placement or at the time of Direct to Implant. The benefit of the mesh and the ADM will be long-lasting due to the device design and can achieve the benefits of the ADM in the upper pole and the strength of the synthetic mesh in the lower pole.

In this device, the ADM is positioned anterior to the most projecting portion of the expander/implant and superiorly—this is where ADM is most useful for coverage, for creation of a plane for fat grafting, and for supporting upper mastectomy flaps. The device's lower and lower lateral pole is covered with a synthetically designed mesh that lasts for approximately two years—offering stronger support where the patient and implant need such. This reinforces the fold and lateral breast in a more permanent way than ADM alone—supporting the pocket, limiting implant descent and malposition, and limiting rippling.

A secondary benefit of such a tight pocket is limiting implant flipping—which is seen more commonly with the transition to higher cohesive implants and more projection higher volumes of such. Pocket control is the success driver in implant-based breast reconstruction, and this dual biosynthetic mesh fills a void in the market that could become the gold standard across the nation and beyond.

The innovation of combining two technologies in this fashion is unexpected because the use of synthetic mesh in the breast pocket is a relatively new technology (becoming more popular over the last 5 years). While ADM in breast has enjoyed decades of data and use in breast reconstruction, surgeons have really only thought of synthetic mesh as a secondary choice or one used to correct complications of failing implant based breast reconstruction (implant mal position, rippling and loss of anatomic landmarks). The idea of combining the two in the devices described herein allows the surgeon to use the benefits of lasting strength in the lower pole of the breast with the benefits of ADM coverage in the upper pole where rippling and fat grafting is more useful.

The ADM for the upper pole of the device could be either human dermis (e.g., MTF, AlloDerm) or animal dermis or animal material (e.g., strattice, bovine or porcine dermis, ovitex, artia, etc.). The unique characteristic of this part of the device is that this area is a scaffold that the patient's own blood vessels and tissue grows into and in theory this product adds thickness to the overall tissue thickness in this area.

The synthetic mesh for the lower pole of the device is intended to harbor strength and more longevity in redefining anatomical landmarks and supporting an implant over time. For this reason, all synthetic options are under consideration, such as vicryl mesh, permanent mesh like gortex, poly(4-Hydroxy buterate) (galaflex), Polydioxanone (PDO, Durasorb), polyglycolic acid trimethylaene carbonate (PGA-PMC Tigr mesh), seri scaffold, etc. In some examples, it is preferable that the lower pole of the mesh would absorb over 2 years. In all cases, the synthetic mesh should allow replacement of themselves overtime with the patient's own collagen. In some examples, it is preferable that the synthetic mesh exhibits a strength profile that allows more than 16 N/cm (e.g., as calculated by Deeken et al consistent with strength needs in the abdomen for hernia patients) (Deeken et al. Journal of mechanical Behavior of Biomedical Materials, 2017, 7, 411-427). The strength of this mesh over time will likely decrease and the 16 N/cm translates to an absolute value of 47.88 Newtons of strength in some ball burst parameter models; accordingly, an initial burst strength at or above 50 N may be required before the patient's own collagen contributes to burst strength success in the future.

The ADM can be connected to the synthetic component of the device using any suitable method. For example, the synthetic mesh can be sewn into the ADM. This area of binding can, in some example, be a single suture line. In some examples, the area of binding can extend to a cross over connection extending from 0.1 centimeter or more depending on strength and union needs.

### Example 2

The problem the devices described herein aim to solve is the lack of technology that can both support breast implants in breast reconstruction and offer soft tissue coverage of the most projecting portion and the upper pole of the implant.

Biologic mesh (e.g., acellular dermal matrix, ADM) has been used for many years in breast reconstruction. Plastic surgeons have used ADM in the breast for more than two decades. The goal is to reinforce damaged soft tissue, support implant in breast reconstruction, supply soft tissue coverage, and redefine landmarks lost at the time of mastectomy. ADM does a relatively good job at achieving those goals, however over time (months), the dermis loses strength, and the lower pole of the breast reconstruction descends as the ADM stress and integrity are lost. That results in the visualized rippling of the implant through the skin (due to loss of support) as well as implant malposition and descent and then eventual reoperations.

The field of breast reconstruction responded with synthetic meshes (e.g., Seri Scaffold, Galaflex, Durasorb) that have a higher strength profile and support the breast fold and implant more aggressively and for a more extended period of time. The synthetic meshes do a better job at keeping the pocket tight and supporting the lower pole, but these synthetic meshes are thin and do not offer soft tissue coverage nor a plane for fat grafting over the implant—as ADMs do. Further, mesh alone constricts and does not allow for tissue expander expansion as needed in two staged breast reconstructions (ADM expands, mesh does not expand significantly).

Each mesh (ADM and Synthetic) is useful in different locations in the breast reconstruction, and the devices described herein comprises a bio-synthetic mesh that combines the strengths of both into one device for breast reconstruction.

The old methods of breast reconstruction were the use of no acellular dermis and simply reconstruction with an implant alone. This was wrought with technical challenges and aesthetic failures. With the addition of ADM, the ability to achieve better outcomes was improved, but over time the implants have descended, and rippling has become a bigger problem due to the limitation of the dermal mesh strength in the lower pole of the breast.

The synthetic meshes that have recently come to market address the strength of the lower pole issue. However, they do not allow expansion with tissue expanders and they do not offer soft tissue coverage in the upper pole of the breast where such is needed.

An advantage of the devices described herein is that it takes the best from ADM and the best from synthetic meshes and combines them into one device to control the pocket in implant-based breast reconstruction.

### Example 3

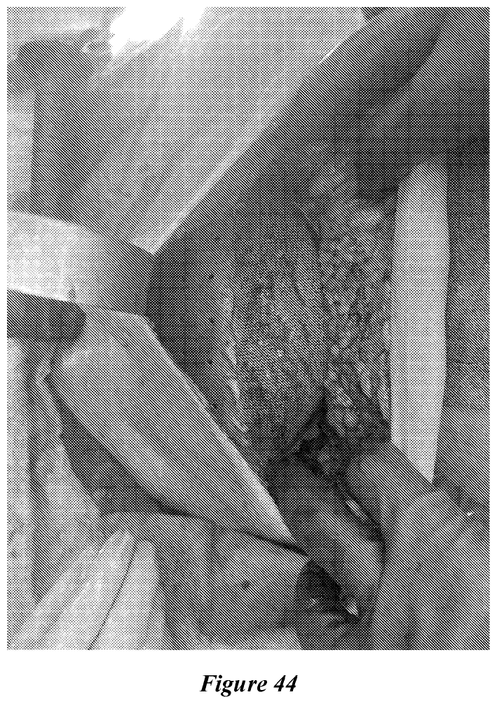

Photographs of example devices are shown in FIG. 44-FIG. 46.

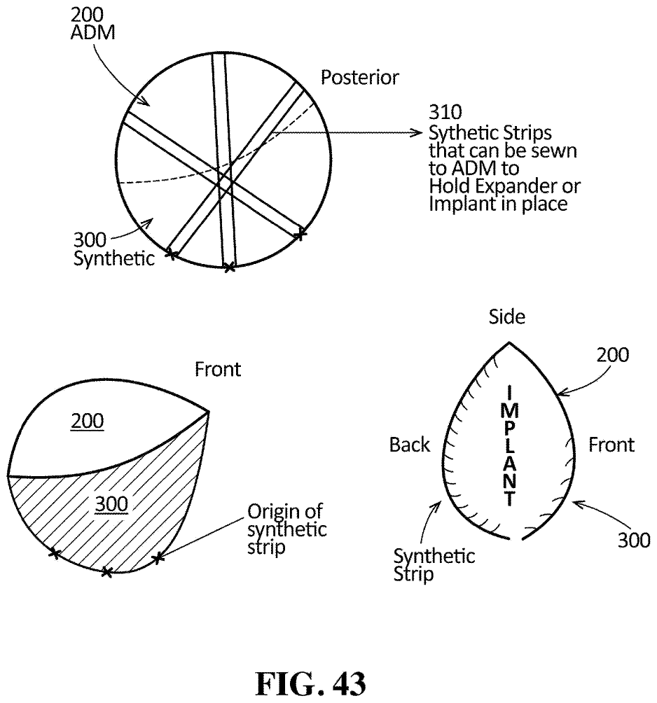

In some examples, the devices can further include a strip of the synthetic mesh sewn to ADM for stability/support to hold implant or expander in place, for example as shown in FIG. 43.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A device for facilitating reconstruction, augmentation, and/or cosmetic improvement of a breast, the breast having an upper pole, a lower pole, a most projecting portion, and an inframammary fold, the device comprising:
a superior portion comprising a biological matrix sheet, the superior portion having a lower end positioned towards the most projecting portion, the superior portion being configured to extend superiorly from the most projecting portion of the breast and form a slope extending along the upper pole of the breast;
an inferior portion comprising a synthetic mesh sheet, the inferior portion having an upper end positioned towards the most projecting portion, the inferior portion being configured to extend inferiorly from the upper end and form an inward curvature extending along the lower pole of the breast towards the inframammary fold; and
a connection portion configured to connect the lower end of the superior portion to the upper end of the inferior portion.

2. The device of claim 1, wherein at least a portion of the connection portion is positioned at or below the most projecting portion of the breast.

3. The device of claim 1, wherein the connection portion is a seam extending between the lower end and the upper end.

4. The device of claim 1, wherein the superior portion is contiguous with the inferior portion along the connection portion.

5. The device of claim 1, wherein the lower end includes a lower edge, the upper end includes an upper edge, and the lower edge is connected to the upper edge to form at least a portion of the connection portion.

6. The device of claim 1, wherein the superior portion and the inferior portion overlap along the connection portion.

7. (canceled)

8. (canceled)

9. (canceled)

10. The device of claim 1, wherein the inferior portion further includes a lateral portion extending at least partially into the upper pole.

11. (canceled)

12. (canceled)

13. (canceled)

14. The device of claim 1, wherein the biological matrix sheet comprises an acellular tissue matrix (ATM).

15. (canceled)

16. (canceled)

17. The device of claim 1, wherein the superior portion is perforated.

18. The device of claim 1, wherein the synthetic mesh sheet is biodegradable.

19. The device of claim 1, wherein the synthetic mesh sheet comprises vicryl mesh, gortex, poly(4-Hydroxybuterate), Polydioxanone (PDO), polyglycolic acid trimethylene carbonate (PGA-PMC), silk, polypropylene and/or polyglycolic acid, or a combination thereof.

20. (canceled)

21. (canceled)

22. The device of claim 1, wherein the device has a surface area, from 40 to 60% of the surface area of the device is the superior portion, from 40 to 60% of the surface area of the device is the inferior portion, the superior portion and the inferior portion together comprising 90% or more of the surface area of the device.

23. The device of claim 1, wherein the device is symmetric in shape.

24. The device of claim 1, wherein the device is asymmetric in shape.

25. (canceled)

26. (canceled)

27. (canceled)

28. The device of claim 1, wherein the device is reversible.

29. (canceled)

30. The device of claim 1, wherein the superior portion, the inferior portion, the device, or a combination thereof is substantially flat.

31. The device of claim 1, wherein the superior portion, the inferior portion, the device, or a combination thereof is cup shaped.

32. The device of claim 1, wherein the device is configured to be utilized with a breast implant or tissue expander.

33. (canceled)

34. (canceled)

35. (canceled)

36. (canceled)

37. The device of claim 32, wherein the superior portion has a first edge, the inferior portion has a second edge, and the device has a periphery comprising at least a portion of the first edge and the second edge, and wherein the device further comprises one or more strips of synthetic material, each of the one or more strips extending from a portion of the second edge to a distal end, wherein the distal end of each of the one or more strips is configured to be attached to a portion of the first edge, for example such that each of the one or more strips can at least partially encircle the breast, breast implant, and/or tissue expander.

38-46. (canceled)

47. A kit comprising the device of claim 1 disposed within a sterile package.

48-59. (canceled)
